(19) United States
(12) Patent Application Publication
(10) Pub. No.: US 2009/0054307 A1
(43) Pub. Date: Feb. 26, 2009

(54) PROPHYLACTIC AND THERAPEUTIC AGENTS AND USES THEREFOR

(75) Inventors: Seong-Seng Tan, Victoria (AU); Sharad Kumar, Victoria (AU)

Correspondence Address: KNOBBE MARTENS OLSON & BEAR LLP, 2040 MAIN STREET, FOURTEENTH FLOOR, IRVINE, CA, 92614, US

(73) Assignee: Howard Florey Institute of Experimental Physiology and Medicine, Parkville, Victoria (AU)

(21) Appl. No.: 12/065,605

(22) PCT Filed: Sep. 1, 2006
(86) PCT No.: PCT/AU06/01283

§ 371 Date: Sep. 2, 2008

Related U.S. Application Data
(60) Provisional application No. 60/713,908, filed on Sep. 1, 2005.

(30) Foreign Application Priority Data
Sep. 1, 2005 (AU) 2005904801

(51) Int. Cl. A61K 31/7088; A61K 38/16; C12Q 1/68; A61K 9/127; A61P 25/00

(52) U.S. Cl. 514/8; 514/44; 435/6; 424/450

(57) ABSTRACT

Treatment, detection and monitoring of disease of the nervous system, especially trauma or hypoxia, in particular in the central nervous system and the eye by up-regulation or increasing levels of Ndfip1 (also known as Nedd4-WW Domain Binding Protein 5 or N4WBP5). Prophylaxis of such conditions in pre-term infants, coronary artery bypass graft, chemotherapy, tumor irradiation and other patients.

### BACKGROUND OF THE INVENTION

1. Field of the Invention

The present invention relates generally to the field of prophylaxis, treatment, detection and monitoring of disease and/or trauma of the nervous system as well as other conditions and to methods useful for same. More particularly, the present invention relates to the identification of Ndfip1 (formally N4WBP5) and its binding partner Nedd4 as neuronal and cellular survival factors, especially following disease and/or trauma. The present invention further provides a medical assessment system in the form of an animal model of acute diseases and traumas of the nervous, respiratory and coronary systems.

2. Description of the Prior Art

Reference to any prior art in this specification is not, and should not be taken as, an acknowledgment or any form of suggestion that this prior art forms part of the common general knowledge in any country.

Bibliographic details of references provided in this document are listed at the end of the specification.

Neurological disorders represent some of the most physically and intellectually debilitating conditions which affect humans. Whilst substantive research has been undertaken on chronic neurological conditions such as in Alzheimer's disease and Parkinson's disease, less is known about the affects of acute injury to the nervous system whether caused by physical trauma or an acute disease condition.

Following traumatic brain injury (TBI), cortico neurons undergo wholesale cellular changes involving inflammation, apoptosis, necrotic cell death, immune response, ischemia and release of free radicals, among others (Faden A I, Curr Opin Neurol 15:707-712, 2002). The transcriptional drivers behind these processes are being keenly elucidated, assisted by gene profiling studies that enable a large number of genes to be simultaneously studied. Typically, these studies are conducted using postmortem tissue obtained 4 to 24 hours after TBI and involve probing mRNA against cDNA arrays or gene chips after TBI and involve probing mRNA against cDNA arrays or gene chips (Kobori, Brain Res Mol Brain Res 104:148-158, 2002; Natale et al, J Neurotrauma 20:907-927, 2003; Rall et al, Neruopathol Appl Nerobiol 29:118-131, 2003; Rao et al, J Neruotrauma 16:865-877, 1999; Yoshiya et al, J Neurotrauma 20:1147-1162, 2003; Keyvani et al, J Neruopathol Exp Neurol 63:598-609, 2004; Di Giovanni et al, Proc Natl Acad Sci USA 102:8333-8338, 2005). Together, these studies have confirmed that the trauma response is characterized by altered transcription of genes related to inflammation, apoptosis, neurotransmitter release, cell-cycle activation, gliosis, reactive oxygen metabolism, ionic homeostasis and neurodegeneration. Of particular interest are genes conferring neuroprotection since the ultimate aim of these studies is to identify candidate molecular pathways as novel targets for therapeutic intervention.

In accordance with the present invention, transcriptional changes are identified in Ndfip1 (formally N4WBP5) providing a therapeutic target for a range of conditions such as in the nervous system (including brain trauma or disease), respiratory system (including hypoxia), coronary system (including coronary bypass grafting or CABG) and ocular system.

### SUMMARY OF THE INVENTION

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Nucleotide and amino acid sequences are referred to by a sequence identifier number (SEQ ID NO). The SEQ ID NOs correspond numerically to the sequence identifiers <400>1 (SEQ ID NO:1), <400>2 (SEQ ID NO:2), etc. A summary of the sequence identifiers is provided in Table 1. A sequence listing is provided at the end of the specification.

Genes are represented herein in italics (e.g. Ndfip1 (formally N4WBP4) or Nedd4). Gene expression products, i.e. mRNA or proteins, are represented in non-italicised form (e.g. Ndfip1 (formally N4WBP5) is the expression product of Ndfip1 (formally N4WBP5) and Nedd4 is the expression product of Nedd4). References to Ndfip1 (formally N4WBP5), Ndfip1 (formally N4WBP5), Nedd4 and Nedd4 include homologs and functional equivalents thereof.

Understanding the transcriptional response of neuronal injury following trauma is a necessary prelude to formulation of therapeutic strategies. In accordance with the present invention, SAGE identified up-regulated genes in the cortex up to 2 hrs following traumatic injury. Biological replication of SAGE data was performed with qRT-PCT using multiple cortical samples following trauma at 2 hrs, 6 hrs, 12 hrs and 24 hrs. This analysis revealed that the vast majority of genes were down-regulated from the 2 hrs timepoint onwards. Further confirmation was obtained by in situ hybridization of a subset of down-regulated genes. Of particular interest was Nedd4 and its adaptor Ndfip1 (formally N4WBP5) which were both strongly expressed above normal background levels in TUNEL-negative neurons surrounding the trauma site. As these proteins are involved in protein ubiquitination, it is proposed herein that neuronal survival following trauma is associated with increased protein ubiquitination.

The present invention relates, therefore, to agents useful for the prophylaxis and treatment of diseases and traumas on the nervous system, respiratory system, coronary system and ocular system and to methods useful for same. More particularly, the agents of the present invention regulate the expression and/or activity of Ndfip1 (formally N4WBP5) and/or Nedd4 or which modulate Ndfip1 (formally N4WBP5)-Nedd4 interaction. The agents of the present invention are useful, inter alia, for preventing or treating or ameliorating the effects of a range of acute neurological diseases and traumatic injuries such as following severe head injuries, trauma-induced paralysis, infection and starvation. The present invention also facilitates the development of diagnostic and/or prognostic assays and reagents useful for identifying an acute disease and/or injury or the severity of a disease and/or injury in the nervous system of a subject. In addition, the diagnostic agents are useful for monitoring a therapeutic protocol. The present invention also facilitates the development of a medical assessment system in the form of an animal model of nervous system acute diseases and/or injuries characterized by abnormal Ndfip1 (formally N4WBP5) or Nedd4 expression and/or Ndfip1 (formally N4WBP5) or Nedd4 activity or interaction.

The present invention, therefore, is predicated in part on the determination that Ndfip1 (formally N4WBP5) and/or Nedd4 expression in neural tissue is increased following acute stress, such as caused by traumatic injury. As such, the present invention provides target genes and gene products which assist in promoting survival of neural cells. It is proposed, therefore, that the prophylaxis and/or treatment of acute diseases and injuries requiring the neuron survival is carried out via increasing levels of expression of Ndfip1 (formally N4WBP5) or Nedd4 or activity or interaction of Ndfip1 (formally N4WBP5) or Nedd4. These proteins and genes are also a useful monitor of the state of health of neurological tissue or the success or otherwise of a therapeutic protocol.

In addition, the Ndfip1 (formally N4WBP5) is associated with protection during coronary artery bypass grafting (CABG), protection prior to, during or following strokes, protection prior to, during or following hypoxia in preterm infants or in the eye or prior to, during or following tumor irradiation or chemotherapy.

In one embodiment, therefore, the present invention provides agents which modulate the expression of Ndfip1 (formally N4WBP5) or Nedd4 or activity or interactability of Ndfip1 (formally N4WBP5) or Nedd4. Alternatively, the agents of the present invention may comprise the activity gene or gene products of Ndfip1 (formally N4WBP5) and/or Nedd4. By “Modulate”, preferably includes up-regulates or otherwise promotes increased levels.

The agents of the present invention may be any proteinaceous molecules such as peptides, polypeptides and proteins or non-proteinaceous molecules such as nucleic acid molecules and small to large natural or synthetically derived organic and inorganic molecules.

In another embodiment, the present invention also provides for methods of identifying agents useful for modulating (i.e. increasing) the level of expression of Ndfip1 (formally N4WBP5) or Nedd4 or level of activity of Ndfip1 (formally N4WBP5) or Nedd4 and thereby promoting neural or other cell survival. These methods of identification comprise screening naturally produced libraries, chemical produced libraries, as well as combinatorial libraries, phage display libraries and in vitro translation-based libraries.

In yet another embodiment, the present invention provides a method of promoting neural or other cell survival, said method comprising contacting a cell with an agent which is capable of up-regulating the level of expression of Ndfip1 (formally N4WBP5) and/or Nedd4 level of activity of Ndfip1 (formally N4WBP5) and/or Nedd4 for a time and under conditions sufficient to promote the survival of neural or other cells.

The agents and methods of the present invention also facilitate the development of methods and pharmaceutical compositions for preventing and/or treating a range of acute neurological diseases and traumatic injuries and/or other conditions in a subject such as, but not limited to head injuries, trauma-induced paralysis, infection, starvation, acute pathogen infection, stroke, hypoxia and/or coronary artery bypass grafting (CABG).

The present invention also facilitates the development of diagnostic and/or prognostic assays and reagents useful for identifying or assessing the presence of an acute disease and/or injury or the severity of an acute disease and/or injury in the nervous or other systems of an subject wherein the disease and/or injury is characterized by an abnormal levels of expression of Ndfip1 (formally N4WBP5) or Nedd4 and/or level of activity of Ndfip1 (formally N4WBP5) or Nedd4.

The present invention provides, therefore, a method of diagnosing and/or prognosing a disease and/or injury characterized by abnormal levels of expression of Ndfip1 (formally N4WBP5) and/or Nedd4 and/or level of activity of Ndfip1 (formally N4WBP5) and/or Nedd4 in the nervous system of a subject said method comprising determining the level of expression of Ndfip1 (formally N4WBP5) and/or Nedd4 and/or level of activity of Ndfip1 (formally N4WBP5) and/or Nedd4 in a biological sample obtained from a subject and determining whether the level of expression of Ndfip1 (formally N4WBP5) and/or Nedd4 and/or level of activity of Ndfip1 (formally N4WBP5) and/or Nedd4 is above or below a threshold level wherein a level of expression of Ndfip1 (formally N4WBP5) and/or Nedd4 and/or level of activity of Ndfip1 (formally N4WBP5) and/or Nedd4 which is above a threshold level is indicative of the presence of a disease and/or injury, or the propensity to develop a disease and/or injury, or the severity of a disease and/or injury in the nervous system of a subject. The term “activity” also includes the interactability of Ndfip1 (formally N4WBP5) and Nedd4. This method is also useful for monitoring a therapeutic protocol.

The present invention also facilitates the development of a medical assessment system in the form of an animal model of nervous or other system diseases and/or injuries characterized by abnormal Ndfip1 (formally N4WBP5) and/or Nedd4 expression and/or Ndfip1 (formally N4WBP5) and/or Nedd4 activity.

TABLE 1
SUMMARY OF SEQUENCE IDENTIFIERS

| SEQUENCE | |
|---|---|
| ID NO: | DESCRIPTION |
| 1 | Nucleotide sequence of human Ndfip1 (formally |
| | N4WBP5) |
| 2 | Amino acid sequence of Ndfip1 (formally N4WBP5) |
| 3 | Nucleotide sequence of murine Nedd4 |
| 4 | Amino acid sequence of murine Nedd4 |

### BRIEF DESCRIPTION OF THE FIGURES

FIG. 1 is a photographic representation showing temporal expression of Ndfip1 (formally N4WBP5) protein after the trauma. A-B″: Double immunostaining with Ndfip1 (formally N4WBP5) (red) and NeuN (green). Low (A) and high (B-B″) power views show that Ndfip1 (formally N4WBP5) immunoreactivity is present in the cytoplasm of all the neurons revealed by NeuN staining. C-I: Ndfip1 (formally N4WBP5) immunostaining (red) combined with TUNEL labeling (green). C: An example at the lesion side 6 hrs after trauma, showing over-expressed Ndfip1 (formally N4WBP5) cells scattered in a band of TUNEL labeled apoptotic cells. D-D″: High power view shows that Ndfip1 (formally N4WBP5) over-expressed cells do not colocalize with TUNEL labeled apoptotic cells (arrows). E-I: Representative images of Ndfip1 (formally N4WBP5) immunstaining and TUNEL labeling at 2 hours sham and on the lesion side at different time after the trauma. E: Section from 2 hours sham, showing no TUNEL labeled cells or Ndfip1 (formally N4WBP5) over-expressed cells. F-I: Images from different time after the trauma, few Ndfip1 (formally N4WBP5) over-expressed neurons are present at 2 hours after the trauma, the number of Ndfip1 (formally N4WBP5) over-expressed cells increases at 6 and reaches its peak at 12 hours after trauma. J: Quantitative analysis of ratio between Ndfip1 (formally N4WBP5) over-expressed cell and TUNEL labeled cells demonstrates significant changes occur at 6 h, 12 hours and 24 hours after trauma. The trend of the ratio change correlates with the mRNA fold changes at different time detected by quantitative real-time PCR. Value represents mean±SEM. Scale bar=100 μm in A (also apples to C), 10 μm in B (also applies to B′, B″, D, D′, D″), 50 μm in E (also applies to F-I). Color photographs are available from the patentee upon request.

FIG. 2 is a photographic representation showing Ndfip1 (formally N4WBP5) and its association with GM130 (A-B″) and Nedd4 (C-D″). A-B″. Double immunostaining with Ndfip1 (formally N4WBP5) (red) and GM130 (green) 6 hrs after trauma. A-A″ on the contralateral side, showing Ndfip1 (formally N4WBP5) is colocalized with GM-130 (a marker for cis Golgi). B-B″ on the lesion side, showing over-expressed Ndfip1 (formally N4WBP5) is colocalized with GM130. In rare occasion, over-expressed Ndfip1 (formally N4WBP5) labeled cell does not colocalize with GM130 (inset, indicated by arrow). C-D″. Double immunostaining with Ndfip1 (formally N4WBP5) (green) and Nedd4 (red) 6 hours after trauma. Ndfip1 (formally N4WBP5) and Nedd4 are present in the same cell (C-C″) on the contralateral side. D-D″, showing over-expressed Nedd4 and Ndfip1 (formally N4WBP5) surrounding the lesion. Scale bar=10 μm in A (applied to all). Color photographs are available from the patentee upon request.

FIG. 3 is a photographic representation showing E13.5 cortical cells cultured for 7 days and transfected with a plasmid coding for pcDNA3-Ndfip1 (formally N4WBP5)-Flag.

FIG. 4 is a diagrammatic representation showing propidium iodine stain and FACs counting following introduction of Ndfip1 (formally N4WBP5) into N18 neuronal cells. Cobalt chloride was used to induce hypoxic conditions. The results show that 34% of cells without Ndfip1 (formally N4WBP5) died whereas only 6% of cells with Ndfip1 (formally N4WBP5) died.

FIG. 5 is a diagrammatic representation showing lentiviral construct which over-expresses Ndfip1 (formally N4WBP5)-GFP.

### DETAILED DESCRIPTION OF THE INVENTION

It is to be understood that unless otherwise indicated, the subject invention is not limited to specific formulation components, manufacturing methods, dosage regimens, or the like, as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context clearly dictates otherwise. Thus, for example, reference to “a neural cell” includes a single neural cell, as well as two or more neural cells; reference to “an agent” includes a single agent, as well as two or more agents; reference to “the gene” includes a single gene or multiple genes; and so forth.

In one embodiment, the present invention provides agents which modulate the expression of a gene or the level of activity of a gene product involved in regulating neural or other cell survival. Alternatively, the agents of the present invention may comprise the gene or gene product involved in promoting neural or other cell survival. In a particular embodiment, the modulation is an up-regulation or promotion of expression or activity.

Reference herein to a “neural cell” means any cell which comprises the nervous system of a subject such as but not limited to a neuron, astrocyte or oligodendrocyte. The term “neural cell” also includes neural stem cells. Reference herein to a neural stem cell should also be taken to include reference- to a “neural precursor cell” or “neural progenitor cell” or any other cell with neural stem cell characteristics. The terms “neural” and “neuronal” are used interchangeably as are neural cells and neurons.

Reference herein to “promoting neural cell survival” includes to increasing the survival rate of a neural cell or population of neuronal cells. In the context of promoting neural cell survival “modulating the expression of a gene or the level of activity of a gene product” preferably means increasing the expression of a gene or the level of activity of a gene product.

Other conditions associated with Ndfip1 (formally N4WBP5) are as follows.

Following coronary artery bypass grafting (CABG), many patients experience both short-term and long-term cognitive impairment. Short-term impairment occurs for up to 3 months after surgery whereas long-term impairment tends to occur 1 to 5 years after surgery. The aetiology is unknown but it is postulate that neuronal death (in the central nervous system) from microemboli and hypoperfusion during CABG is a major contributor. In accordance with the present invention, ischemic injury to neurons is prevented or ameliorated by up-regulation of Ndfip1 (formally N4WBP5) prior to, during and after CABG. Up-regulation of Ndfip1 (formally N4WBP5), or an agent that produces this up-regulation, is neuroprotective as a prophylactic measure administered to the patient.

Data show that Ndfip1 (formally N4WBP5) is over-expressed in surviving neurons following brain ischemia induced by endothelin injection to occlude the middle cerebral artery in rates. Neurons that up-regulate Ndfip1 (formally N4WBP5) do not stain for TUNEL, an indicator of cell death. Ndfip1 (formally N4WBP5) is over-expressed in these surviving neurons from as early as 12 hours and extending to 72 hours. Hence, Ndfip1 (formally N4WBP5) is neuroprotective.

Preterm children who develop severe chronic lung disease (bronchopulmonary dysplasia) are developmentally compromised by exposure to hypoxic episodes. Chronic hypoxia affects the developing brain and contributes to increased neuronal death during the critical period of synaptogenesis and pruning. In humans, this leads to long-term impairments in visual-motor, gross and fine motor, articulation, reading, mathematics, spatial memory and attention skills.

Ndfip1 (formally N4WBP5) protects, therefore, against neuronal death from hypoxic episodes in preterm infants and children if Ndfip1 (formally N4WBP5) (or a mimetic thereof) is introduced or its gene up-regulated in neurons.

The retina, containing photoreceptors is very sensitive to oxygen levels. Hence, diseases that cause low levels of oxygen in the blood since heart, lung and diabetic diseases cause retinal hypoxia. This leads to retinal diseases such as von Hippel-Lindau, retinitis pigmentosa, proliferative diabetic retinopathy, retinopathy of prematurity and glaucoma. Based on its action in the brain, increased Ndfip1 (formally N4WBP5) protects neurons in the retina, particularly the rod and con photoreceptors from injury and death in these conditions.

During ionizing irradiation of the brain to treat brain tumors in young children, there is collateral damage causing death of normal neurons. Over-expression of Ndfip1 (formally N4WBP5) in these situations increases the survival of irradiated neurons but not part of the tumor.

An increase in this regard refers to a 1 to 1000% increase such as a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 64, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500 or 1000% increase.

In a preferred aspect of the present invention, the gene or gene product involved in promoting neural cell survival is Ndfip1 (formally N4WBP5) and Nedd4 or their respective gene products, Ndfip1 (formally N4WBP5) and Nedd4. Reference herein to “Ndfip1 (formally N4WBP5)” and “Nedd4” refers to a nucleic acid sequence that encodes Ndfip1 (formally N4WBP5) and Nedd4, respectively such as a nucleic sequence comprising SEQ ID NOs:1 and 3 or a nucleotide sequence having at least 60% identity to SEQ ID NOs:1 and 3 or a nucleotide sequence capable of hybridizing to SEQ ID NOs:1 and 3 or its complement under low stringency conditions. Reference herein to “Ndfip1 (formally N4WBP5)” and “Nedd4” should also be understood as including reference to all forms of these genes such as homologs, paralogs, orthologs, derivatives, fragments, mimetics, functional equivalents and any nucleic acid sequence that hybridizes to Ndfip1 (formally N4WBP5) and/or Nedd4. Similarly, reference herein to Ndfip1 (formally N4WBP5) or Nedd4 refers to an amino acid sequence such as an amino acid sequence comprising SEQ ID NOs:2 and 4 or an amino acid sequence having about 60% similarity to SEQ ID NOs:2 and 4. Reference herein to “Ndfip1 (formally N4WBP5)” or “Nedd4” should also be understood as including reference to all forms of these proteins such as homologs, paralogs, orthologs, derivatives, fragments, mimetics and functional equivalents thereof.

The terms “agent”, “compound”, “active agent”, “pharmacologically active agent”, “medicament”, “active” and “drug” may be used interchangeably herein to refer to a substance that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “agent”, “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The agents of the present invention may be any proteinaceous molecules such as peptides, polypeptides and proteins or non-proteinaceous molecules such as nucleic acid molecules and small to large natural or synthetically derived organic and inorganic molecules.

As described hereinbefore, the agents of the present invention may be any proteinaceous molecules such as peptides, polypeptides and proteins. In relation to proteinaceous molecules, including peptides, polypeptide and proteins, without distinction, the terms mutant, part, derivative, homolog, analog or mimetic are meant to encompass alternative forms of the agent which promote neural cell survival.

Mutant forms may be naturally occurring or artificially generated variants of Ndfip1 (formally N4WBP5) or Nedd4 or Ndfip1 (formally N4WBP5) or Nedd4 comprising one or more amino acid substitutions, deletions or additions. Mutants may be induced by mutagenesis or other chemical methods or generated recombinantly or synthetically. Alanine scanning is a useful technique for identifying important amino acids (Wells, Methods Enzymol 202:2699-2705, 1991). In this technique, an amino acid residue is replaced by Alanine and its effect on the peptide's activity is determined. Each of the amino acid residues of the peptide is analyzed in this manner to determine the important regions of the polypeptide. Mutants are tested for their ability to regulate angiogenesis and for other qualities such as longevity, binding affinity, dissociation rate and ability to cross biological membranes.

Parts of the agents of the present invention may encompass sections of a full-length agent which is involved in regulating neural cell survival, such as but not limited to Ndfip1 (formally N4WBP5) and/or Nedd4. Sections are at least 10, preferably at least 20 and more preferably at least 30 contiguous amino acids, which exhibit the requisite activity. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of an amino acid sequence of the invention with proteinases such as endoLys-C, endoArg-C, endoGlu-C and staphylococcus V8-protease. The digested fragments can be purified by, for example, high performance liquid chromatographic (HPLC) techniques. Any such part, section or fragment, irrespective of its means of generation, is also to be understood as being encompassed by the term “derivative” as used herein.

Thus derivatives, or the singular derivative, encompass parts, mutants, homologs, fragments, analogues as well as hybrid or fusion molecules and glycosylaton variants. Derivatives also include molecules having a percent amino acid sequence identity over a window of comparison after optimal alignment. Preferably, the percentage similarity between a particular sequence and a reference sequence is at least about 60% or at least about 70% or at least about 80% or at least about 90% or at least about 95% or above such as at least about 96%, 97%, 98%, 99% or greater. Preferably, the percentage similarity between species, functional or structural homologs of the instant agents is at least about 60% or at least about 70% or at least about 80% or at least about 90% or at least about 95% or above such as at least about 96%, 97%, 98%, 99% or greater. Percentage similarities or identities between 60% and 100% are also contemplated such as 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

Analogs of the agents contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecule or their analogs. This term also does not exclude modifications of the polypeptide, for example, glycosylations, acetylations, phosphorylations and the like. Included within the definition are, for example, polypeptides containing one or more analogs of an amino acid (including, for example, unnatural amino acids such as those given in Table 2) or polypeptides with substituted linkages. Such polypeptides may need to be able to enter the cell and/or cross the blood-brain barrier.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids, contemplated herein is shown in Table 2.

TABLE 2
CODES FOR NON-CONVENTIONAL AMINO ACIDS

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-a-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-y-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbaraylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Mimetics are another useful group of agents for regulating neural cell survival. The term is intended to refer to a substance which has some chemical similarity to the molecule it mimics but which antagonizes or agonizes its interaction with a target, such as, for example, Ndfip1 (formally N4WBP5) and/or Nedd4. A peptide mimetic may be a peptide-containing molecule that mimics elements of protein secondary structure (Johnson et al., Peptide Turn Mimetics in Biotechnology and Pharmacy, Pezzuto et al., Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic, therefore, is designed to permit molecular interactions similar to the natural molecule.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. As described hereinbefore, Alanine scans of peptides are commonly used to refine such peptide motifs. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of a receptor and ligand are modelled. This can be especially useful where the receptor and/or ligand change conformation on binding, allowing the model to take account of this in the design of the mimetic. Modelling can be used to generate agents which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, for example, enhance or interfere with the function of a polypeptide in vivo (see e.g. Hodgson, BioTechnology 9:19-21, 1991). In one approach, one first determines the three-dimensional structure of a protein of interest by x-ray crystallography, by computer modelling or most typically, by a combination of approaches. Useful information regarding the structure of a polypeptide may also be gained by modelling based on the structure of homologous proteins.

The present invention also contemplates immunointeractive molecules, particularly antibodies, specific for one or more of the target gene expression products, i.e. Ndfip1 (formally N4WBP5) and/or Nedd4. In the context of this aspect of the present invention, the target gene expression product is an antigen.

The term “antigen” is used herein in its broadest sense to refer to a substance that is capable of reacting in and/or inducing an immune response. Reference to an “antigen” includes an antigenic determinant or epitope. By “antibody” is meant a protein of the immunoglobulin family that is capable of combining, interacting or otherwise associating with an antigen. An antibody is, therefore, an antigen-binding agent or an “immunointeractive agent”. Any agent that has binding affinity for a target antigen is referred to as an immunointeractive agent. It will be understood that this term extends to immunoglobulins (e.g. polyclonal or monoclonal antibodies), immunoglobulin fragments and non-immunoglobulin derived protein frameworks that exhibit antigen-binding activity. The terms “immunointeractive agent” and “antibody” include deimmunized forms of these molecules. An “antibody” is, therefore, an example of an immunointeractive agent and includes a polyclonal or monoclonal antibody. An antibody includes parts thereof including Fab portions and antigen-binding determinants.

The term “immunoglobulin” is used herein to refer to a protein consisting of one or more polypeptides substantially encoded by immunoglobulin genes. The recognized immunoglobulin genes include the κ, λ, α, γ (IgG₁, IgG₂, IgG₃, IgG₄), δ, ε and μ constant region genes, as well as the myriad of other immunoglobulin variable region genes. One form of immunoglobulin constitutes the basic structural unit of an antibody. This form is a tetramer and consists of two identical pairs of immunoglobulin chains, each pair having one light and one heavy chain. In each pair, the light and heavy chain variable regions (V_{L} and V_{H} respectively) are together responsible for binding to an antigen, and the constant regions are responsible for the antibody effector functions. In addition to antibodies, immunoglobulins may exist in a variety of other forms including, for example, Fv, scFv, Fab, Fab′ and (Fab′)₂.

That part of an antigen against which a particular immune response is directed is referred to as an “antigenic determinant” or “epitope” and includes a hapten. Typically, in an animal, antigens present several or even many epitopes simultaneously. A “hapten” is a substance that can combine specificity with an antibody but cannot or only poorly induces an immune response unless bound to a carrier. A hapten typically comprises a single antigenic determinant or epitope.

In relation to polyclonal antibodies, immunization and subsequent production of antibodies may be done using any methods known to those of skill in the art. Similarly, for monoclonal antibody production, immunization and subsequent production of antibodies may also be done using any methods known to those of skill in the art, e.g. Köhler and Milstein (Nature 256:495-499, 1975; Köhler and Milstein, Eur J Immunol 6:511-519, 1976), Coligan et al (Current Protocols in Immunology, John Wiley & Sons, Inc., 1991-1997) or Toyama et al (“Monoclonal Antibody, Experiment Manual”, published by Kodansha Scientific, 1987). Essentially, an animal is immunized with an antigen-containing biological fluid or fraction thereof by standard methods to produce antibody-producing cells, particularly antibody-producing somatic cells (e.g. B-lymphocytes, splenocytes). These cells can then be removed from the immunized animal for immortalization. The antigen may need to first be associated with a larger molecule. The latter is any substance of typically high molecular weight to which a non- or poorly immunogenic substance (e.g. a hapten) is naturally or artificially linked to enhance its immunogenicity.

Immortalization of antibody-producing cells may be carried out using methods, which are well known in the art. For example, the immortalization may be achieved by the transformation method using Epstein-Barr virus (EBV) (Kozbor et al, Methods in Enzymology 121:140-167, 1986). In a preferred embodiment, antibody-producing cells are immortalized using the cell fusion method (described in Coligan et al, 1991-1997, supra), which is widely employed for the production of monoclonal antibodies. In this method, somatic antibody-producing cells with the potential to produce antibodies, particularly B cells, are fused with a myeloma cell line. These somatic cells may be derived from the lymph nodes, spleens and peripheral blood of primed animals, preferably rodent animals such as mice and rats. In the exemplary embodiment of this invention mice, spleen cells are used. The use, however, of rat, rabbit, sheep and goat cells, or cells from other animal species is also contemplated.

Specialized myeloma cell lines have been developed from lymphocytic tumors for use in hybridoma-producing fusion procedures (Köhler and Milstein, 1976 supra; Shulman et al, Nature 276:269-270, 1978; Volk et al, J Virol 42:220-227, 1982). These cell lines have been developed for at least three reasons. The first is to facilitate the selection of fused hybridomas from unfused and similarly indefinitely self-propagating myeloma cells. Usually, this is accomplished by using myelomas with enzyme deficiencies that render them incapable of growing in certain selective media that support the growth of hybridomas. The second reason arises from the inherent ability of lymphocytic tumor cells to produce their own antibodies. To eliminate the production of tumor cell antibodies by the hybridomas, myeloma cell lines incapable of producing endogenous light or heavy immunoglobulin chains are used. A third reason for selection of these cell lines is for their suitability and efficiency for fusion.

Many myeloma cell lines may be used for the production of fused cell hybrids, including, e.g. P3X63-Ag8, P3X63-AG8.653, P3/NS1-Ag4-1 (NS-1), Sp2/0-Ag14 and S194/5.XXO.Bu.1. The P3X63-Ag8 and NS-1 cell lines have been described by Köhler and Milstein 1976 supra. Shulman et al 1978 supra, developed the Sp2/0-Ag14 myeloma line. The S194/5.XXO.Bu.I line was reported by Trowbridge (J Exp Med 148:313-323, 1978).

Methods for generating hybrids of antibody-producing spleen or lymph node cells and myeloma cells usually involve mixing somatic cells with myeloma cells in a 10:1 proportion (although the proportion may vary from about 20:1 to about 1:1), respectively, in the presence of an agent or agents (chemical, viral or electrical) that promotes the fusion of cell membranes. Fusion methods have been described (Köhler and Milstein, Nature 256:495-499, 1975; Köhler and Milstein, Eur J Immunol 6:511-519, 1976; Gefter et al, Somatic Cell Genet. 3:231-236, 1977; Volk et al, J Virol 42:220-227, 1982). The fusion-promoting agents used by those investigators were Sendai virus and polyethylene glycol (PEG).

Because fusion procedures produce viable hybrids at very low frequency (e.g. when spleens are used as a source of somatic cells, only one hybrid is obtained for roughly every 1×10⁵ spleen cells), it is preferable to have a means of selecting the fused cell hybrids from the remaining unfused cells, particularly the unfused myeloma cells. A means of detecting the desired antibody-producing hybridomas among other resulting fused cell hybrids is also necessary. Generally, the selection of fused cell hybrids is accomplished by culturing the cells in media that support the growth of hybridomas but prevent the growth of the unfused myeloma cells, which normally would go on dividing indefinitely. The-somatic cells used in the fusion do not maintain long-term viability in vitro culture and hence do not pose a problem. In the example of the present invention, myeloma cells lacking hypoxanthine phosphoribosyl transferase (HPRT-negative) were used. Selection against these cells is made in hypoxanthine/aminopterin/thymidine (HAT) medium, a medium in which the fused cell hybrids survive due to the HPRT-positive genotype of the spleen cells. The use of myeloma cells with different genetic deficiencies (drug sensitivities, etc.) that can be selected against in media supporting the growth of genotypically competent hybrids is also possible.

Several weeks are required to selectively culture the fused cell hybrids. Early in this time period, it is necessary to identify those hybrids which produce the desired antibody, so that they may subsequently be cloned and propagated. Generally, around 10% of the hybrids obtained produce the desired antibody, although a range of from about 1 to about 30% is not uncommon. The detection of antibody-producing hybrids can be achieved by any one of several standard assay methods, including enzyme-linked immunoassay and radioimmunoassay techniques as, for example, described in Kennet et al ((eds) Monoclonal Antibodies and Hybridomas: A New Dimension in Biological Analyses, pp. 376-384, Plenum Press, New York, 1980). In a particularly preferred embodiment, an enzyme linked immunosorbent assay (ELISA) is performed to select the desired anti-idiotypic antibody-producing clones.

Once the desired fused cell hybrids have been selected and cloned into individual antibody-producing cell lines, each cell line may be propagated in either of two standard ways. A suspension of the hybridoma cells can be injected into a histocompatible animal. The injected animal will then develop tumors that secrete the specific monoclonal antibody produced by the fused cell hybrid. The body fluids of the animal, such as serum or ascites fluid, can be tapped to provide monoclonal antibodies in high concentration. Alternatively, the individual cell lines may be propagated in vitro in laboratory culture vessels. The culture medium containing high concentrations of a single specific monoclonal antibody can be harvested by decantation, filtration or centrifugation, and subsequently purified.

The cell lines are tested for their specificity to detect the antigen of interest by any suitable immunodetection means. For example, cell lines can be aliquoted into a number of wells and incubated and the supernatant from each well is analyzed by enzyme-linked immunosorbent assay (ELISA), indirect fluorescent antibody technique, or the like. The cell line(s) producing a monoclonal antibody capable of recognizing the target idiotype but which does not recognize non-target antigens or epitopes are identified and then directly cultured in vitro or injected into a histocompatible animal to form tumors and to produce, collect and purify the required antibodies.

Non-animal cells such as a plant, yeast and/or microbial cells may also be used to generate, typically, single-chain antibodies. In this embodiment, such cells are engineered to express nucleic acid molecules which encode a chain of an antibody.

In a preferred aspect, the monoclonal antibodies of the present invention are deimmunized for use in humans. However, the subject invention also extends to antibodies from any source and deimmunized for use in any host. Examples of animal sources and hosts include, but are not limited to, humans and non-human primates (e.g. guerilla, macaque, marmoset), livestock animals (e.g. sheep, cow, horse, donkey, pig), companion animals (e.g. dog, cat), laboratory test animals (e.g. mouse, rabbit, rat, guinea pig, hamster), captive wild animals (e.g. fox, deer), reptiles or amphibians (e.g. cane toad), fish (e.g. zebrafish) and other organisms (e.g. C. elegans). The deimmunized antibodies or part thereof may also be generated in non-animal sources, such as but not limited to, plants. In this regard, and as noted hereinbefore, plants are particularly useful as a source of “plantibodies” such as single chain antibodies.

Antibodies are deimmunized by being subjected to a deimmunization means. Such a process may take any of a number of forms including the preparation of “chimeric” antibodies which have the same or similar specificity as the monoclonal antibodies prepared according to the present invention. Chimeric antibodies are antibodies whose light and heavy chain genes have been constructed, typically by genetic engineering, from immunoglobulin variable and constant region genes belonging to different species. Thus, in accordance with the present invention, once a hybridoma producing the desired monoclonal antibody is obtained, techniques are used to produce interspecific monoclonal antibodies wherein the binding region of one species is combined with a non-binding region of the antibody of another species (Liu et al, Proc Natl Acad Sci USA 84:3439-3443, 1987). For example, the complementary determining regions (CDRs) from a non-human (e.g. murine) monoclonal antibody can be grafted onto a human antibody, thereby “humanizing” the murine antibody (European Patent Publication No. 0 239 400; Jones et al, Nature 321:522-525, 1986; Verhoeyen et al, Science 239:1534-1536, 1988; Riechmann et al, Nature 332:323-327, 1988). In this case, the deimmunizing process is specific for humans. More particularly, the CDRs can be grafted onto a human antibody variable region with or without human constant regions. The non-human antibody providing the CDRs is typically referred to as the “donor” and the human antibody providing the framework is typically referred to as the “acceptor”. Constant regions need not be present, but if they are, they must be substantially identical to human immunoglobulin constant regions, i.e. at least about 85-90%, preferably about 95% or more identical. Hence, all parts of a humanized antibody, except possibly the CDRs, are substantially identical to corresponding parts of natural human immunoglobulin sequences. Thus, a “humanized antibody” is an antibody comprising a humanized light chain and a humanized heavy chain immunoglobulin. A donor antibody is said to be “humanized”, by the process of “humanization”, because the resultant humanized antibody is expected to bind to the same antigen as the donor antibody that provides the CDRs. Reference herein to “humanized” includes reference to an antibody deimmunized to a particular host, in this case, a human host.

Exemplary methods which may be employed to produce deimmunized antibodies according to the present invention are described, for example, in Riechmann et al, Nature 332:323-327, 1988; U.S. Pat. Nos. 6,056,957, 6,180,370 and 6,180,377 and Chothia et al, J Mol Biol 196:901-917, 1987.

As used herein, the term “CDR” includes CDR structural loops which covers to the three light chain and the three heavy chain regions in the variable portion of an antibody framework region which bridge β strands on the binding portion of the molecule. These loops have characteristic canonical structures (Chothia et al, J Mol Biol 227:799-817, 1992; Kabat et al, “Sequences of Proteins of Immunological Interest”, U.S. Department of Health and Human Services, 1983).

In the context of the present invention, the term “heavy chain variable region” means a polypeptide which is from about 110 to 125 amino acid residues in length, the amino acid sequence of which corresponds to that of a heavy chain of a monoclonal antibody of the invention, starting from the amino-terminal (N-terminal) amino acid residue of the heavy chain. Likewise, the term “light chain variable region” means a polypeptide which is from about 95 to 130 amino acid residues in length, the amino acid sequence of which corresponds to that of a light chain of a monoclonal antibody of the invention, starting from the N-terminal amino acid residue of the light chain. Full-length immunoglobulin “light chains” (about 25 Kd or 214 amino acids) are encoded by a variable region gene at the NH₂-terminus (about 110 amino acids) and a κ or λ constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g. γ (encoding about 330 amino acids).

An immunoglobulin light or heavy chain variable region, which is interrupted by three hypervariable regions, also called CDRs, is referred to herein as a “framework region”. The extent of the framework region and CDRs have been precisely defined. The sequences of the framework regions of different light or heavy chains are relatively conserved within a species. As used herein, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDRs. The CDRs are primarily responsible for binding to an epitope of an antigen.

One preferred deimmunization process referred to herein is variable region grafting and results in a “chimeric” antibody. The resulting antibody comprises one or more amino acid substitutions within the v-region when compared to the present (e.g. murine) antibody. The rationale for making v-region changes is to further the potential for an induced immune response in the intended host (e.g. a human). The basis of deimmunization is predicated in part on the assumption that a substantive immune response to an introduced antibody requires a T-cell mediated response. The trigger for the T-cell response is the presentation of processed peptides emanating from the introduced antibody on the surface of antigen presenting cells (APCs). The APCs present such peptides in association with surface MHC class II molecules. The deimmunized approach is, therefore, based on:—
(i) predicting peptide sequences capable of associating with MHC class II molecules; and
(ii) changing strategic residues to eliminate the ability of the peptide to associate with the MHC class II molecule.

The invention also contemplates the generation and use of fragments of monoclonal antibodies produced by the method of the present invention including, for example, Fv, scFv, Fab, Fab′ and F(ab′)₂ fragments. Such fragments may be prepared by standard methods as for example described by Coligan et al (Current Protocols in Immunology, John Wiley & Sons, Inc., 1991-1997).

The present invention also contemplates synthetic or recombinant antigen-binding molecules with the same or similar specificity as the antibodies of the invention. Antigen binding molecules of this type may comprise a synthetic stabilized Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Krebber et al (J Immunol Methods 201:35-55, 1997). Alternatively, they may be prepared by methods described in U.S. Pat. No. 5,091,513, European Patent No 239,400 or the articles by Winter and Milstein (Nature 349:293-299, 1991) and Plückthun et al (In Antibody engineering: A practical approach 203-252, 1996).

Alternatively, the synthetic stabilised Fv fragment comprises a disulphide stabilized Fv (dsFv) in which cysteine residues are introduced into the V_{H} and V_{L} domains such that in the fully folded Fv molecule the two residues will form a disulphide bond there between. Suitable methods of producing dsFv are described, for example, in (Glockshuber et al, Biochem 29:1363-1367, 1990; Reiter et al, J Biol Chem 269:18327-18331, 1994; Reiter et al, Biochem 33:5451-5459, 1994; Reiter et al, Cancer Res 54:2714-2718, 1994; Webber et al, Mol Immunol 32:249-258, 1995).

Also contemplated as synthetic or recombinant antigen-binding molecules are single variable region domains (termed dAbs) as, for example, disclosed in (Ward et al, Nature 341:544-546, 1989; Hamers-Casterman et al, Nature 363:446-448, 1993; Davies and Riechmann, FEBS Lett 339:285-290, 1994).

Alternatively, the synthetic or recombinant antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the synthetic or recombinant antigen binding molecule may comprise non-immunoglobulin derived, protein frameworks. For example, reference may be made to Ku and Schutz (Proc Natl Acad Sci USA 92:6552-6556, 1995) which discloses a four-helix bundle protein cytochrome b562 having two loops randomized to create CDRs, which have been selected for antigen binding.

The synthetic or recombinant antigen-binding molecule may be multivalent (i.e. having more than one antigen binding site). Such multivalent molecules may be specific for one or more antigens. Multivalent molecules of this type may be prepared by dimerization of two antibody fragments through a cysteinyl-containing peptide as, for example disclosed by (Adams et al, Cancer Res 53:4026-4034, 1993; Cumber et al, J Immunol 149:120-126, 1992). Alternatively, dimerization may be facilitated by fusion of the antibody fragments to amphiphilic helices that naturally dimerize (Plünckthun, Biochem 31:1579-1584, 1992) or by use of domains (such as leucine zippers jun and fos) that preferentially heterodimerize (Kostelny et al, J Immunol 148:1547-1553, 1992). In further embodiment, a multi-step process is employed such as first administering a deimmunized antibody and then an anti-antibody with, for example, a reporter molecule.

The present invention further encompasses chemical analogs of amino acids in the deimmunized murine monoclonal antibodies described herein. The use of chemical analogs of amino acids is useful inter alia to stabilize the deimmunized murine monoclonal antibodies when administered to a subject. As described hereinbefore, the analogs of the amino acids contemplated herein include, but are not limited to, modifications of side chains, incorporation of unnatural amino acids and/or their derivatives during peptide, polypeptide or protein synthesis and the use of crosslinkers and other methods which impose conformational constraints on the proteinaceous molecule or their analogs.

As noted hereinbefore, the agents of the present invention may also be nucleic acid molecules. As such, the present invention also extends to a genetic approach for up-regulating the expression of Ndfip1 (formally N4WBP5) and Nedd4 or the level of activity of Ndfip1 (formally N4WBP5) and/or Nedd4 or the interactability between Ndfip1 (formally N4WBP5) and Nedd4. This could involve, inter alia, providing gene function to cell such as in a gene therapy, or, it could involve inhibiting the function or a gene in which its expression product down-regulates expression of Ndfip1 formally N4WBP5) or Nedd4 or down-regulates the activity of Ndfip1 (formally N4WBP5) and/or Nedd4 using gene silencing constructs and antisense oligonucleotides.

A target nucleic acid sequence or a part of a nucleic acid sequence, such as the nucleic acid sequences identified in Table 1, i.e. SEQ ID NOs:1, 2, 3 and/or 4, may be introduced into a cell in a vector such that the nucleic acid sequence remains extrachromosomal. In such a situation, the nucleic acid sequence is expressed by the cell from the extrachromosomal location. Vectors for introduction of nucleic acid sequence both for recombination and for extrachromosomal maintenance are known in the art and any suitable vector may be used. Methods for introducing nucleic acids into cells such as electroporation, calcium phosphate co-precipitation and viral transduction are known in the art.

In particular, a number of viruses have been used as nucleic acid transfer vectors or as the basis for preparing nucleic acid transfer vectors, including papovaviruses (e.g. SV40, Madzak et al, J Gen Virol 73:1533-1536, 1992), adenovirus (Berkner, Curr Top Microbiol Immunol 158:39-66, 1992; Berkner et al, BioTechniques 6:616-629, 1988; Gorziglia and Kapikian, J Virol 66:4407-4412, 1992; Quantin et al, Proc Natl Acad Sci USA 89:2581-2584, 1992; Rosenfeld et al, Cell 68:143-155, 1992; Wilkinson et al, Nucleic Acids Res 20:233-2239, 1992; Stratford-Perricaudet et al, Hum Gene Ther 1:241-256, 1990; Schneider et al, Nat Genetics 18:180-183, 1998), vaccinia virus (Moss, Curr Top Microbiol Immunol 158: 5-38, 1992; Moss, Proc Natl Acad Sci USA 93:11341-11348, 1996), adeno-associated virus (Muzyczka, Curr Top Microbiol Immunol 158:97-129, 1992; Ohi et al, Gene 89:279-282, 1990; Russell and Hirata, Nat Genetics 18:323-328, 1998), herpesviruses including HSV and EBV (Margolskee, Curr Top Microbiol Immunol 158:67-95, 1992; Johnson et al, J Virol 66:2952-2965, 1992; Fink et al, Hum Gene Ther 3:1-19, 1992; Breakefield and Geller, Mol Neurobiol 1:339-371, 1987; Freese et al, Biochem Pharmaco. 40:2189-2199, 1990; Fink et al, Ann Rev Neurosci 19:265-287, 1996), lentiviruses (Naldini et al, Science 272:263-267, 1996), Sindbis and Semliki Forest virus (Berglund et al, Biotechnology 11:916-920, 1993) and retroviruses of avian (Bandyopadhyay and Temin, Mol Cell Biol 4:749-754, 1984; Petropoulos et al, J Virol 66:3391-3397, 1992), murine (Miller, Curr Top Microbiol Immunol 158:1-24, 1992; Miller et al, Mol Cell Biol 5:431-437, 1985; Sorge et al, Mol Cell Biol 4:1730-1737, 1984; Mann and Baltimore, J Virol 54:401-407, 1985; Miller et al, J Virol 62:4337-4345, 1988) and human (Shimada et al, J Clin Invest 88:1043-1047, 1991; Helseth et al, J Virol 64:2416-2420, 1990; Page et al, J Virol 64:5270-5276, 1990; Buchschacher and Panganiban, J Virol 66:2731-2739, 1982) origin.

Non-viral nucleic acid transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated nucleic acid transfer can be combined with direct in vivo nucleic acid transfer using liposome delivery, allowing one to direct the viral vectors to particular cells. Alternatively, the retroviral vector producer cell line can be injected into particular tissue. Injection of producer cells would then provide a continuous source of vector particles.

In relation to nucleic acid molecules, the terms mutant, section, derivative, homolog, analog or mimetic have analogous meanings to the meanings ascribed to these forms in relation to proteinaceous molecules. In all cases, variant forms are tested for their ability to function as proposed herein using techniques which are set forth herein or which are selected from techniques which are currently well known in the art.

When in nucleic acid form, a derivative comprises a sequence of nucleotides having at least 60% identity to a parent molecule, such as a nucleic acid sequence encoding a binding partner of the present invention, or a section thereof. A “section” of a nucleic acid molecule is defined as having a minimal size of at least about 5 nucleotides or preferably about 10 nucleotides or more preferably at least about 15 nucleotides. This definition includes all sizes in the range of 5-15 nucleotides including 5, 6, 7, 8, 9, 10, 11, 12, 13, 14 or 15, nucleotides as well as greater than 15 nucleotides including 50, 100, 300, 500, 1000 or 2000 nucleotides or nucleic acid molecules having any number of nucleotides within these values. Having at least about 60% identity means, having optimal alignment, a nucleic acid molecule comprises at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identity with a reference sequence which encodes a binding partner of the present invention.

The terms “similarity” or “identity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and amino acid sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al (Nucl Acids Res 25:3389-3402, 1997). A detailed discussion of sequence analysis can be found in Unit 19.3 of Ausubel et al (“Current Protocols in Molecular Biology” John Wiley & Sons Inc, 1994-1998, Chapter 15).

The terms “sequence similarity” and “sequence identity” as used herein refer to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e. the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

The nucleic acid molecules of the present invention are also capable of hybridizing to other genetic molecules. Reference herein to “hybridizes” refers to the process by which a nucleic acid strand joins with a complementary strand through base pairing. Hybridization reactions can be sensitive and selective so that a particular sequence of interest can be identified even in samples in which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low).

Reference herein to a “low stringency” includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as “medium stringency”, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or “high stringency”, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur and Doty, J Mol Biol 5:109-118, 1962). However, the Tₘ of a duplex nucleic acid molecule decreases by 1° C. with every increase of 1% in the number of mismatch base pairs (Bonner and Laskey, Eur J Biochem 46:83-88, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The terms “nucleic acid”, “nucleotide” and “polynucleotide” include RNA (mRNA, tRNA, rRNA, siRNA), DNA (genomic DNA, cDNA), synthetic forms and mixed polymers, both sense and/or antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

In another embodiment, the present invention also provides for methods of identifying agents useful for modulating the level of expression of Ndfip1 (formally N4WBP5) and/or Nedd4 or level of activity of N4WPB5 and/or Nedd4 and promoting neural or other cell survival. These methods of identification comprise screening naturally produced libraries, chemical produced libraries, as well as combinatorial libraries, phage display libraries and in vitro translation-based libraries. The capability of the agents of the present invention, whether they be proteinaceous or non-proteinaceous, to modulate the expression of Ndfip1 (formally N4WBP5) and/or Nedd4 or the level of activity of Ndfip1 (formally N4WBP5) and/or Nedd4 and/or to promote neural cell survival may be assessed via a number of screening methods which would be well known to a person skilled in the art. One method of screening utilizes eukaryotic or prokaryotic host cells which are stably transformed with recombinant polynucleotides expressing a target protein of interest, such as Ndfip1 (formally N4WBP5) and/or Nedd4, preferably in competitive binding assays. Such cells, either in viable or fixed form, can be used for standard binding assays. One may measure, for example, the formation of complexes between a target and the agent being tested, or examine the degree to which the formation of a complex between a target and a known ligand is aided or interfered with by the agent being tested.

The screening procedure includes assaying (i) for the presence of a complex between the agent and the target, or (ii) an alteration in the expression levels of nucleic acid molecules encoding the target. As described hereinbefore, one form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the agent rather than the target.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for agents capable of modulating the level of expression of Ndfip1 (formally N4WBP5) and/or Nedd4 or level of activity of Ndfip1 (formally N4WBP5) and/or Nedd4.

The identification of agents could also be carried out in accordance with the present invention by a process comprising the following steps:
(i) isolating a sample of neural cells or tissue;
(ii) placing samples of the cells or tissue into suitable receptacles; and
(iii) exposing the samples of cells or tissue to agents for a particular period of time and under particular conditions; and
(iv) screening for morphological, physiological and genetic changes to the cells or tissue which are characteristic of regulated neural cell survival.

Two-hybrid screening is also useful in identifying other members of a biochemical or genetic pathway associated with a target. Two-hybrid screening conveniently uses Saccharomyces cerevisiae and Saccharomyces pombe. Target interactions and screens for agonists and antagonists can be carried out using the yeast two-hybrid system, which takes advantage of transcriptional factors that are composed of two physically separable, functional domains. The most commonly used is the yeast GAL4 transcriptional activator consisting of a DNA binding domain and a transcriptional activation domain. Two different cloning vectors are used to generate separate fusions of the GAL4 domains to genes encoding potential binding proteins. The fusion proteins are co-expressed, targeted to the nucleus and if interactions occur, activation of a reporter gene (e.g. lacZ) produces a detectable phenotype. In the present case, for example, S. cerevisiae is co-transformed with a library or vector expressing a cDNA GAL4 activation domain fusion, and a vector expressing a target gene fused to GAL4. If lacZ is used as the reporter gene, co-expression of the fusion proteins will produce a blue color. Small molecules or other candidate compounds which interact with a target will result in loss of color of the cells. Reference may be made to the yeast two-hybrid systems as disclosed by Munder et al. (Appl Microbiol Biotechnol 52:311-320, 1999) and Young et al, Nat Biotechnol 16:946-950, 1998). Molecules thus identified by this system are then re-tested in animal cells.

In yet another embodiment, the present invention provides a method of promoting neural cell survival, said method comprising contacting a neural or other cell with an agent which is capable of increasing the level of expression of Ndfip1 (formally N4WBP5) and/or Nedd4 or level of activity of Ndfip1 (formally N4WBP5) and/or Nedd4 for a time and under conditions sufficient to promote the survival of the neural or other cell.

The agents and methods of the present invention also facilitate the development of methods and pharmaceutical compositions for preventing and/or treating a range of acute neurological diseases and injuries or other conditions in a subject such as, but not limited to head or brain injury, trauma-induced paralysis, infection and starvation by a pathogen (microorganism or virus), hypoxia (in preterm infants and in the eye), protecting subjects following irradiation or chemotherapy of tumors and protecting subjects from CABG.

Reference herein to “treatment” may mean a reduction in the severity of an existing disease or condition. The term “treatment” is also taken to encompass “prophylactic treatment” to prevent the onset of a disease or condition. The term “treatment” does not necessarily imply that a subject is treated until total recovery. Similarly, “prophylactic treatment” does not necessarily mean that the subject will not eventually contract a disease or condition.

Subject as used herein refers to humans and non-human primates (e.g. guerilla, macaque, marmoset), livestock animals (e.g. sheep, cow, horse, donkey, pig), companion animals (e.g. dog, cat), laboratory test animals (e.g. mouse, rabbit, rat, guinea pig, hamster), captive wild animals (e.g. fox, deer), reptiles or amphibians (e.g. cane toad), fish (e.g. zebrafish) and any other organisms (e.g. C. elegans) who can benefit from the agents of the present invention. There is no limitation on the type of animal that could benefit from the presently described agents. The most preferred subject of the present invention is a human. A subject regardless of whether it is a human or non-human organism may be referred to as a patient, individual, animal, host or recipient.

The agents of the present invention can be combined with one or more pharmaceutically acceptable carriers and/or diluents to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., Remington's Pharmaceutical Sciences, 18^{th} Edition, Mack Publishing Company, Easton, Pa., 1990 (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the modulatory agent of the invention and on its particular physio-chemical characteristics.

Administration of the agent, in the form of a pharmaceutical composition, may be performed by any convenient means known to one skilled in the art including parenteral and non-parenteral routes. Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally via inhalation, orally, rectally, patch and implant.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Due to their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier, see, e.g, International Patent Publication Number WO 96/11698.

Agents of the present invention, when administered orally, may be protected from digestion. This can be accomplished either by complexing the agent with a composition to render it resistant to acidic and enzymatic hydrolysis or by packaging the agent in an appropriately resistant carrier such as a liposome. Means of protecting compounds from digestion are well known in the art, see, e.g. Fix, Pharm Res 13:1760-1764, 1996; Samanen et al, J Pharm Pharmacol 48:119-135, 1996; U.S. Pat. No. 5,391,377, describing lipid compositions for oral delivery of therapeutic agents.

The pharmaceutical forms suitable for injectable use include sterile aqueous solutions (where water-soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the agents in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

For parenteral administration, the agent may dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the agents are being administered intrathecally, they may also be dissolved in cerebrospinal fluid.

For transmucosal or transdermal administration, penetrants appropriate to the barrier to be permeated can be used for delivering the agent. Such penetrants are generally known in the art e.g. for transmucosal administration, bile salts and fusidic acid derivatives. In addition, detergents can be used to facilitate permeation. Transmucosal administration can be through nasal sprays or using suppositories e.g. Sayani and Chien, Crit Rev Ther Drug Carrier Syst 13:85-184, 1996. For topical, transdermal administration, the agents are formulated into ointments, creams, salves, powders and gels. Transdermal delivery systems can also include patches.

For inhalation, the agents of the invention can be delivered using any system known in the art, including dry powder aerosols, liquids delivery systems, air jet nebulizers, propellant systems, and the like, see, e.g., Patton, Nat Biotech 16:141-143, 1998; product and inhalation delivery systems for polypeptide macromolecules by, e.g., Dura Pharmaceuticals (San Diego, Calif.), Aradigm (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, for example, air jet nebulizers.

The agents of the invention can also be administered in sustained delivery or sustained release mechanisms, which can deliver the formulation internally. For example, biodegradeable microspheres or capsules or other biodegradeable polymer configurations capable of sustained delivery of an agent can be included in the formulations of the invention (e.g. Putney and Burke, Nat Biotech 16:153-157, 1998).

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes), carbohydrates, or synthetic polymers. For a general discussion of pharmacokinetics, see, e.g., Remington's.

In one aspect, the pharmaceutical formulations comprising agents of the present invention are incorporated in lipid monolayers or bilayers such as liposomes, see, e.g., U.S. Pat. Nos. 6,110,490; 6,096,716; 5,283,185 and 5,279,833. The invention also provides formulations in which water-soluble modulatory agents of the invention have been attached to the surface of the monolayer or bilayer. For example, peptides can be attached to hydrazide-PEG-(distearoylphosphatidyl)ethanolamine-containing liposomes (e.g. Zalipsky et al, Bioconjug Chem 6:705-708, 1995). Liposomes or any form of lipid membrane, such as planar lipid membranes or the cell membrane of an intact cell e.g. a red blood cell, can be used. Liposomal formulations can be by any means, including administration intravenously, transdermally (Vutla et al, J Pharm Sci 85:5-8, 1996), transmucosally, or orally. The invention also provides pharmaceutical preparations in which the agents of the invention are incorporated within micelles and/or liposomes (Suntres and Shek, J Pharm Pharmacol 46:23-28, 1994; Woodle et al, Pharm Res 9:260-265, 1992). Liposomes and liposomal formulations can be prepared according to standard methods and are also well known in the art see, e.g., Remington's; Akimaru et al, Cytokines Mol Ther 1:197-210, 1995; Alving et al, J Immunol Rev 145:5-31, 1995; Szoka and Papahadjopoulos, Ann Rev Biophys Bioeng 9:467-508, 1980, U.S. Pat. Nos. 4,235,871, 4,501,728 and 4,837,028.

The pharmaceutical compositions of the invention can be administered in a variety of unit dosage forms depending upon the method of administration. Dosages for typical pharmaceutical compositions are well known to those of skill in the art. Such dosages are typically advisorial in nature and are adjusted depending on the particular therapeutic context, patient tolerance, etc. The amount of agent adequate to accomplish this is defined as the “effective amount”. The dosage schedule and effective amounts for this use, i.e., the “dosing regimen” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., Remington's; Egleton and Davis, Peptides 18:1431-1439, 1997; Langer, Science 249:1527-1533, 1990.

In accordance with these methods, the agents and/or pharmaceutical compositions defined in accordance with the present invention may be co-administered with one or more other agents. Reference herein to “co-administered” means simultaneous administration in the same formulation or in two different formulations via the same or different routes or sequential administration by the same or different routes. Reference herein to “sequential” administration is meant a time difference of from seconds, minutes, hours or days between the administration of the two types of agents and/or pharmaceutical compositions. Co-administration of the agents and/or pharmaceutical compositions may occur in any order.

Alternatively, targeting therapies may be used to deliver the active agent more specifically to certain types of cell, by the use of targeting systems such as, but not limited to, antibodies or cell specific ligands. Targeting may be desirable for a variety of reasons, e.g. if the agent is unacceptably toxic or if it would otherwise require too high a dosage or if it would not otherwise be able to enter the target cells, e.g., by not being able to cross the blood-brain barrier. The inability to cross the blood-brain barrier is a particular problem for agents directed to the nervous system, especially the central nervous system, and as such, a number of strategies are well known in the art for improving the accessibility of the central nervous system to administered agents (Misra et al, J Pharm Sci 6:252-273, 2003).

The present invention also facilitates the development of diagnostic and/or prognostic assays and reagents useful for identifying the presence of a disease and/or injury, or the propensity to develop a disease and/or injury, or the severity of a disease and/or injury in the nervous or other system of an subject wherein the disease and/or condition is characterized by an abnormal levels of expression of Ndfip1 (formally N4WBP5) and/or Nedd4 and/or level of activity of Ndfip1 (formally N4WBP5) and/or Nedd4.

The present invention provides, therefore, a method of diagnosing and/or prognosing a disease and/or injury characterized by abnormal level of expression of Ndfip1 (formally N4WBP5) and/or Nedd4 and/or level of activity of Ndfip1 (formally N4WBP5) and/or Nedd4 in the nervous or other system of a subject said method comprising determining the level of expression of Ndfip1 (formally N4WBP5) and/or Nedd4 and/or level of activity of Ndfip1 (formally N4WBP5) and/or Nedd4 in a biological sample obtained from a subject and determining whether the level of expression of Ndfip1 (formally N4WBP5) and/or Nedd4 and/or level of activity of Ndfip1 (formally N4WBP5) and/or Nedd4 is above or below a threshold level wherein a level of expression of Ndfip1 (formally N4WBP5) and/or Nedd4 and/or level of activity of Ndfip1 (formally N4WBP5) and/or Nedd4 which is above a threshold level is indicative of the presence of a disease and/or injury, or the propensity to develop a disease and/or injury, or the severity of a disease and/or injury in the nervous or other system of a subject.

Reference herein to “biological sample” includes any biological sample obtained from a subject. Examples of suitable samples include those obtained from cells, a biological fluid (such as blood, plasma, serum, urine, bile, saliva, tears, cerebrospinal fluid, aqueous or vitreous humor, or any bodily secretion).

Samples may also be obtained from any organ or tissue (including a biopsy or autopsy specimen) or may comprise cells (including primary cells, passaged or cultured primary cells, cell lines, cells conditioned by a specific medium) or medium conditioned by cells. In preferred embodiments, a biological sample is free of intact cells. If desired, the biological sample may be subjected to prior processing, such as lysis, extraction, subcellular fractionation, and the like, see, e.g., Deutscher (Ed), Methods Enzymol 182:147-238, 1990.

The present invention also facilitates the development of a medical assessment system in the form of an animal model of nervous system diseases and/or injuries characterized by abnormal Ndfip1 (formally N4WBP5) and/or Nedd4 expression and/or Ndfip1 (formally N4WBP5) and/or Nedd4 activity.

The animal models of the present invention are preferably genetically modified organisms.

Reference herein to a “genetically modified organism” refers to an organism that contains within its genome a specific gene that has been modified. Modification to a gene occurs, inter alia, when the nucleic acid sequence comprising the gene is disrupted and/or mutated. Disruption and mutation may comprise single or multiple nucleic acid insertions, deletions, substitutions or combinations thereof. Disruption and/or mutation in a gene may, for example, alter the normal expression of the gene by enhancing or inhibiting (partially or totally) the expression of the RNA and protein which the gene encodes.

The genetically modified organism of the present invention may be a non-human primate (e.g. guerilla, macaque, marmoset), livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer, horse, donkey), reptile or amphibian (e.g. cane toad), fish (e.g. zebrafish) or any other organism (e.g. C. elegans). Preferably the genetically modified organism is a mouse.

Techniques for constructing genetically modified organisms are well known in the art (see, e.g., Hogan et al., Manipulating the Mouse Embryo: A Laboratory Manual, Cold Spring Harbor Laboratory Press, Cold Spring Harbor N.Y., 1986; Robertson (Ed), Teratocarcinomas and Embryonic Stem Cells: A Practical Approach. IRL Press, Washington D.C., 1987; Mansour et al., Nature 336:348-352, 1988; Capecchi et al., Trends Genet 5:70-76, 1989, Capecchi et al., Science 244:1288-1292, 1989; Pickert, Transgenic Animal Technology: A Laboratory Handbook. Academic Press, San Diego, Calif., 1994).

In generating the genetically modified organism of the present invention a targeting construct may be used. Reference herein to a “targeting construct” refers to an artificially constructed segment of genetic material which can be transferred into selected cells. The targeting construct can integrate with the genome of the host cell in such a position so as to enhance or inhibit (partially or entirely) expression of a specific gene.

The targeting construct may be produced using standard methods known in the art (e.g. Sambrook and Russell, Molecular Cloning: A Laboratory Manual, 3^{rd} Edition, Cold Spring Harbor Laboratory Press, Cold Spring Harbor, N.Y., 2001; Ausubel (Ed), Current Protocols in Molecular Biology, 5^{th} Edition, John Wiley & Sons, Inc, NY, 2002).

The targeting construct of the present invention may also comprise a positive selection marker. Examples of selectable markers include genes conferring resistance to compounds such as antibiotics, genes conferring the ability to grow on selected substrates, genes encoding proteins that produce detectable signals such as luminescence. A wide variety of such markers are known and available, including, for example, antibiotic resistance genes such as the neomycin resistance gene (neo) and the hygromycin resistance gene (hyg). Selectable markers also include genes conferring the ability to grow on certain media substrates such as the tk gene (thymidine kinase) or the hprt gene (hypoxanthine phosphoribosyltransferase) which, confer the ability to grow on HAT medium (hypoxanthine, aminopterin and thymidine); and the bacterial gpt gene (guanine/xanthine phosphoribosyltransferase) which allows growth on MAX medium (mycophenolic acid, adenine and xanthine). Other selectable markers for use in mammalian cells and plasmids carrying a variety of selectable markers are well known in the art.

The preferred location of the marker gene in the targeting construct will depend on the aim of the gene targeting. For example, if the aim is to inhibit target gene expression, then the selectable marker can be cloned into targeting DNA corresponding to coding sequence in the target gene. Alternatively, if the aim is to express an altered product from the target gene or to enhance expression of the target gene, then the selectable marker can be placed outside of the coding region, for example, in a nearby intron.

The selectable marker may depend on its own promoter for expression and the marker gene may be derived from a very different organism than the organism being targeted (e.g. prokaryotic marker genes used in targeting mammalian cells). However, it is preferable to replace the original promoter with transcriptional machinery known to function in the recipient cells. A large number of transcriptional initiation regions are available for such purposes including, for example, metallothionein promoters, thymidine kinase promoters, β-actin promoters, immunoglobulin promoters, SV40 promoters and human cytomegalovirus promoters. A widely used example is the pSV2-neo plasmid which has the bacterial neomycin phosphotransferase gene under control of the SV40 early promoter and confers in mammalian cells resistance to G418 (an antibiotic related to neomycin). A number of other variations may be employed to enhance expression of the selectable markers in animal cells, such as the addition of a poly(A) sequence and the addition of synthetic translation initiation sequences. Both constitutive and inducible promoters may be used.

The targeting construct of the present invention may also comprise loxP and frt sites to facilitate site specific recombination in the presence of cre and flp recombinase respectively.

The development of the targeting construct of the present invention facilitates its introduction into a host cell. Reference herein to a “host cell” includes an individual cell or cell population that can be or has been a recipient for the incorporation of nucleic acid molecules. Host cells include progeny of a single host cell, and the progeny may not necessarily be genetically identical to the original parent due to natural, accidental or deliberate mutation. A host cell includes those cells transfected with the targeting constructs of the present invention.

A host cell in the context of the present invention is preferably derived from a non-human primate (e.g. guerilla, macaque, marmoset), livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer, horse, donkey), reptile or amphibian (e.g. cane toad), fish (e.g. zebrafish) or any other organism (e.g. C. elegans). In the most preferred embodiment of the invention the host cell is derived from a mouse.

Various techniques for introducing a targeting construct into a host cell, either in vivo or in vitro, are well known in the art and include, but are not limited to, microinjection, viral-mediated transfer and electroporation. In a preferred embodiment of the present invention, the targeting construct is introduced into the host cell by electroporation. In the electroporation process, electrical impulses of high field strength reversibly permeabilize biomembranes allowing the introduction of the construct into the host cell. The pores created during electroporation permit the uptake of macromolecules such a nucleic acids (Potter et al., Proc Natl Acad Sci U.S.A. 81:7161-7165, 1984).

The host cell of the present invention can be any host cell whose genome is capable of homologous recombination. Reference herein to “homologous recombination” refers to the exchange of nucleic acid regions between two nucleic acid molecules at the site of homologous nucleotide sequences.

The present invention contemplates stem cells or embryonic stem (ES) cells or embryonic cells or embryos for use in generating an organism which produces substantially higher levels of Ndfip1 (formally N4WBP5) and/or Nedd4.

The preferred host cell of the present invention is an ES cell which is typically derived from pre-implantation embryos maintained in vitro (see, e.g., Evans et al., Nature 292:154-156, 1981; Bradely et al., Nature 309:255-258, 1984; Gossler et al., Proc Natl Acad Sci U.S.A. 83:9065-9069, 1986 and Robertson et al., Nature 322:445-448, 1986). The ES cells are cultured and prepared for introduction of the targeting construct using methods well known to a person skilled in the art (see, e.g., Hogan et al., supra; Robertson (Ed), supra). The ES cells that will be inserted with the targeting construct are derived from an embryo or blastocyst of the same species as the developing embryo into which they are to be introduced. ES cells are typically selected for their ability to integrate into the inner cell mass and contribute to the germ line of an individual when introduced into the mammal in an embryo at the blastocyst stage of development. Thus, any ES cell line having this capability is suitable for use in the practice of the present invention

After the targeting construct has been introduced into the host cells, the cells in which successful gene targeting has occurred are identified. Insertion of the targeting construct into the targeted gene is typically detected by identifying cells for expression of the marker gene as described hereinbefore. In a preferred embodiment, the cells transformed with the targeting construct of the present invention are subjected to treatment with an appropriate agent that selects against cells not expressing the selectable marker. Only those cells expressing the selectable marker gene survive and/or grow under certain conditions.

Successful recombination may be identified by analyzing the DNA of the selected host cells to confirm homologous recombination. Various techniques known in the art, such as PCR and/or Southern analysis may be used to confirm homologous recombination events. Selected host cells that have undergone successful homologous recombination are then injected into a blastocyst (or other stage of development suitable for the purposes of creating a viable organism, such as, for example, a morula) to form chimeras. Alternatively, selected ES cells can be allowed to aggregate with dissociated embryo cells to form the aggregation chimera. A chimeric embryo can then be implanted into a suitable pseudopregnant female foster organism and the embryo brought to term. Chimeric progeny harboring the homologously recombined DNA in their germ cells can be used to breed organisms in which all cells of the organism contain the homologously recombined nucleic acid. In one embodiment, chimeric progeny mice are used to generate an organism with a heterozygous modification in one allele of the Ndfip1 (formally N4WBP5) gene or the Nedd4 gene. Heterozygous genetically modified organisms can then be interbred. It is well known in the art that typically 25% the offspring of such matings will have a homozygous modification to both alleles of one or both genes.

The heterozygous and homozygous genetically modified organism of the present invention can then be compared to a non-genetically modified organism of the same species to determine whether a mutant target causes changes in the phenotype of the genetically modified organism. Reference herein to “phenotype” should be understood as a reference to the totality of the characteristics, or any particular characteristic or set of characteristics, of a cell and/or organism as determined by interaction of the genotype of the cell and/or organism with the environment in which it exists.

In one embodiment, the genetically modified organism of the present invention produces substantially higher levels of Ndfip1 (formally N4WBP5) and/or Nedd4.

The genetically modified organism of the present invention may be in the form of the mature organism or may be, for example, in the form of the immature organism (e.g. embryos) for transplantation. The immature organism is preferably maintained in a frozen state and may optionally be sold with instructions for use.

It should also be understood that the present invention also provides a genetically modified cell comprising the targeting construct described hereinbefore. These cells may be derived from any suitable source, such as the genetically modified organism described hereinbefore, or may be generated by any suitable means, such as the means described hereinbefore for introducing a targeting construct into a host cell. Such cells include stem cells and embryonic cells which are preferably maintained in a frozen state and may be sold for use in generating an organism which produces substantially higher levels of Ndfip1 (formally N4WBP5) and/or Nedd4.

The present invention is further described by the following non-limiting examples.

### EXAMPLE 1

### Materials and Methods

Part of this information is published in Sang et al, The Journal of Neuroscience 26(27):7234-7244, 2006 which is incorporated herein by reference.

Experimental brain injury. Cortical trauma was induced using an experimental model for closed head injury as previously described (Chen, 1996). In brief, twelve-week old male C57BL/6 mice were ether-anaesthetized and their skulls exposed by a longitudinal incision of the scalp. Trauma was initiated to a localized region in the left cerebral hemisphere 2 mm lateral to the midline in the midcoronal plane. An electric weight-drop device was attached with a metal rod of 333 g falling from 2 cm above the cortical surface. The tip of the rod was coated with a silicone tip of 3 mm diameter to prevent penetrating skull fractures. After the procedure, the scalp incision was closed and the animals allowed to recover. Sham-operated animals were anaesthetized and their scalps exposed followed by closure.

Long SAGE libraries. Following TBI or sham treatment, animals were killed with ether overdose at 2 h. Brains were removed and left cortical hemispheres dissected from the underlying structures. Total RNA was isolated from pooled left cortices (n=6, each group) using Trizol (Invitrogen Life Technology, Carlsbad, Calif.). LongSAGE libraries were constructed according to SAGE protocol Version B (1-SAGE (Trademark) Long Kit, Invitrogen Life Technology, Carlsbad, Calif.) (Saha, 2002). SAGE tag extraction was performed by using either the program SAGE2000 (www.sagenet.org) or specifically-developed software from DNA sequence output files (Applied Biosystems). All tags representing linkers were removed. Tag identities were matched to genes using the Refseq database in the National Center for Biotechnology Information (www.ncbi.nlm.nih.gov).

Statistical Analysis of SAGE data. Fisher's exact test was used to identify differentially expressed genes between trauma and sham-treated cortical libraries. Given the counts for a certain tag and the sum of all tags present in the libraries, Fisher's exact test computed the P value that these counts could have been observed by chance if the tag was equally represented in the libraries under comparison. The Benjamini and Hochberg correction was used to control the false-discovery rate associated with a large-scale multiple-testing environment (Benjamini, 1995). Tags with a corrected P value <0.10 were considered to be significant and were differentially expressed between libraries. More details may be found at www.mbgproiect.org/fisher.test.html.

SAGE Tag Mapping to Genes and Chromosomal Positions. A comparative chromosomal view of expression levels in TBI and sham-treated cortices was generated by assigning chromosomal positions for tags corresponding to known genes after matching to the RefSeq database at the National Centre for Biotechnology Information (February 2003 release; Mm3 at http://genome.ucsc.edu/). Where a tag matches several genes, a unique identity was assigned using the ranking priority by the RefSeq database: NM (curated mRNA)>XM (not-curated, mRNA). In cases of multiple matches within the same category, no identity was assigned. Tag counts were normalized to 100,000 tags per library. To correlate tag abundances with gene-poor and gene-rich chromosomal regions, in silico reconstruction of Giemsa bands in the mouse genome was carried out using the methodology previously applied for the human genome (Niimura, 2002). To detect differential expression between the two tissues, a series of data points was generated (at log₂ scale) to depict the ratio of expression levels between the two tissues. By using a 10-Mb window, all tags present for TBI (or sham-operated) were summed to yield a data point at the midpoint of the 10-Mb window. Serial data points for each library were generated along each chromosome by sliding the window forward by one tag position at a time. To compute for the significance of these ratios, an identical process was performed on a randomized gene order 10,000 times.

Quantitative real-time PCR (qRT-PCR). Genes that were statistically identified by Fishers exact test to be differentially-expressed were independently verified using qRT-PCR. The relative abundance of mRNA levels at various time-points after TBI (2 hours, 6 hours, 12 hours and 24 hours) and sham-control (2 hours) were examined. Eighteen genes were processed on ABI 7700 sequence-detection system and 94 genes were processed using low density arrays (Applied Biosystems). Primers for ABI 7700 detection system were designed using Primer Express (Applied Biosystems). Total RNA was isolated from the left cortex at 2 hrs following sham-treatment (n=5); or 2 hours (n=5), 6 hours (n=3), 12 hours (n=3) or 24 hours (n=3) following TBI using RNeasy kit (QIAGEN Sciences, MD). For the 2 hours time point, real-time PCR was also performed using total RNA that was used to construct the SAGE libraries. 1 μg of total RNA was DNase treated and 0.3 μg was reverse-transcribed to cDNA using Taqman Reverse Transcription Reagents (Applied Biosystems, Foster City, Calif.). qRT-PCR using Sybr green chemistry (Applied Biosystems, Foster City, Calif.) was performed on ABI 7700 sequence detection system. Endogenous 18S ribosomal RNA was used as an internal reference. For low density arrays, a 7900HT custom-made Micro Fluidic Card Configuration 7 (containing 94 genes and two endogenous controls—18s and GAPDH) was used to quantify mRNA levels following the manufacturers' protocol (Applied Biosystems, Foster City, Calif.). The cycle number at which the fluorescence emission exceeds the fixed threshold was defined as threshold cycle (C_{T}). ΔC_{T} value was the C_{T} value of the gene of interest substracted by C_{T} value for 18S. ΔΔC_{T} value was obtained by subtracting of the mean value of ΔC_{T} obtained from 2 hours sham tissues which served as calibator from the ΔC_{T} of traumatic tissues. The equation of 2_{T}^{-ΔΔC} was used to obtain the fold change of the mRNA level of the interested gene of the traumatic tissues relative to the mRNA level of 2 hours sham. Statistic analysis was performed with the One-Way Anova Test.

In situ hybridisation and immunohistochemistry. A number of genes were selected for spatial detection of mRNA levels by in situ hybridization and/or immunohistochemistry at 6 hours after TBI or sham-treatment. cDNA image clones were transcribed with either T3, T7 or SP polymerase (Promega, Madison, Wis.) and riboprobes labeled with digoxigenin-11-d-UTP (Roche Diagnostics, City, Germany). Coronal sections (10 μm) were obtained from fresh frozen brains (6 hours after TBI or sham-treatment) and fixed with 4% v/v paraformaldehyde in phosphate-buffered saline for 10 min and acetylated for 10 min before prehybridization for 2 hours in hybridisation buffer (50% v/v formamide, 5× murine sodium citrate (SSC), 5×Denhardt's, 250 μg/ml tRNA and 500 μg/ml herring sperm DNA) at room temperature. Hybridization (1 μg/ml buffer) was carried out overnight at 55° C. Excess probe was removed with 2×SSC at 72° C. for 2 hrs followed by incubation overnight in anti-digoxigenin-AP (1:1500, Roche Diagnostics, city, country). Alkaline phosphatase activity was revealed by nitribule tetrazolium (1 mg/ml, Roche Diagnostic, city, country) and 5-bromo-4-chloro-3-inodolyl phosphate (0.2 mg/ml, Roche Diagnostic, city, country).

Immunohistochemistry was performed on coronal sections (10 μm) obtained from fresh frozen brains collected at 2 hours, 6 hours, 12 hours or 24 hours after TBI or sham-treatment. Following fixation with 4% paraformaldehyde in phosphate-buffered saline, sections were incubated overnight in primary antibodies. All primary antibodies were diluted in 0.1M PBS with 0.3% v/v Triton X-100. Primary antibodies included rabbit polyclonal antibodies raised to Sez6 protein (1:500) (manuscript in preparation); a purified rabbit polyclonal antibody raised to Ndfip1 (formally N4WBP5)-GST fusion protein (1:100) (Harvey, 2002); a purified rabbit polyclonal antibody raised to Nedd4 (dilution and ref); a mouse monoclonal antibody to NeuN (Chemicon, Temecula, Calif.; 1:200); a mouse monoclonal antibody to the Golgi marker, GM130 (BD Transduction Laboratory, San Diego, Calif.; 1:100). To detect apoptotic cells, TUNEL (TdT-mediated dUTP nick end labeling) staining was carried out according to the manufacturer's instructions (Roche Diagnostic, city, country). Secondary antibodies were biotinylated anti-rabbit IgG (Vector Laboratories, Burlingame, Calif.; 1:200); and Alexa Fluor (Trademark) 594 conjugated goat anti-rabbit IgG (Molecular Probes, Eugene, Oreg.; 1:500); FITC-conjugated donkey anti-mouse IgG (Jackson ImmunoResearch, West Grove, Pa.; 1:500).

Since Nedd4 and Ndfip1 (formally N4WBP5) antisera were both raised in rabbits, tyramide signal amplication (TSA; Renaissance Kit, NEN Life Science, Boston, Mass.) was performed to allow both antigens to be visualized in the same cell. Brief explanation of TSA. In brief, the section was blocked with TNB blocking buffer for 30 min before incubating in a rabbit anti-Ndfip1 (formally N4WBP5) (1:5000). The section was then incubated in a biotinylated goat anti-rabbit IgG (1:200, Vector Laboratories, Burlingame, Calif.) for 1 hour and streptavidin-horseradish peroxidase (SA-HRP) for 30 min. The signal was then amplified with biotinyl tyramide and the immunoreactivity revealed by fluorescein-avidin (1:200, Vector Laboratories, Burlingame, Calif.). Double-labeling using rabbit anti-Nedd4 (1:100) was also performed on these sections, and staining revealed with secondary antibody.

### In Vitro Cell Transfection and Growth Factor Deprivation of Neurons.

C57BL/6/J pregnant mice were killed by cervical dislocation. Mouse embyros at embryonic day 13.5 were removed by Caesarean section, and the brain was dissected. Strips of cortical tissue were dissected and placed in 0.1% trypsin in PBS for 30 min at 37° C. The cells were further dissociated by passing the suspension up and down a pipette before suspension in DMEM (Invitrogen) supplemented with 10% fetal calf serum. The cells were plated on a poly-_{D}-lysine (Sigma, St. Louis, Mo.) and laminin (Invitrogen) coated coverslips in a 24-well plate with 200,000 cells in 40 μl of medium per well and cultured with 5% CO₂ at 37° C. overnight before the medium was replaced by Neurobasal medium (Invitrogen) supplemented with 0.5 mM _{L}-glutamine, 1×B27 supplement (Invitrogen), 50 U/ml penicillin, and 50 μg/ml streptomycin. After 3 d culture, the cells were washed with Neurobasal medium without antibiotics and transfected with one of the following plasmids: pcDNA-Flag-E12, pcDNA3-N4WBP5-Flag, pEF-N-Flag-Bcl-2 or pEF-CrmA (cytokine response modifier A)-N-Flag using Lipofectamine 2000 according to the instructions of the manufacturer (Invitrogen). In brief, 0.8 μg of cDNA and 2 μl of Lipofectamine 2000 were separately diluted in 50 μl of Opti-MEM I reduced serum medium and incubated for 5 min before being mixed together and incubated for another 20 min. The complex was then added to each well and incubated for 6 hours at 37° C. with 5% CO₂. To deprive neurons of growth factors, the medium was replaced with Neurobasal medium lacking supplements and cultured for 18 hours before being fixed in 4% paraformaldehyde in 0.1 _{M}PBS for 2 hours. Control cultures received the normal growth supplements. Transfected cells were visualized by immunostaining for the fusion Flag reporter using a mouse anti-Flag antibody (1:1000; Sigma, Sydney, Australia). Double staining with TUNEL was performed to score the percentage of apoptotic cells expressing the Flag reporter. To reveal the ratio of TUNEL-positive cells to all cells in culture, bisbenzamide (1 μg/ml) was used to reveal cell nuclei. Three separate sets of experiments for each condition was performed, the neurons were tallied by an investigator blinded to the experimental conditions, and the final results were pooled together for statistical analysis.

### EXAMPLE 2

### Mapping of Gene Identities and Expression Levels to Chromosomal Positions

Following the removal of linker sequences and mitochondiral genes, a total of 50,760 (sham-control) and 52,476 (TBI) long SAGE tags were obtained from each pool of mRNA obtained 2 hrs after treatment. These tags correlated to roughly 18,000 different genes. A genome-scale view of these transcriptomes can be portrayed by mapping the gene positions and gene densities for all tags from the two libraries onto an in silico map of GC-rich and GC-poor bands of the mouse genome, generated by us according to the method of Niimura and Gojobori (Niimura Y, Gojobori T, Proc Natl Acad Sci USA 99:797-802, 2002). This map is based on similar human studies that predicts, in silico, chromosomal regions of Giemsa-light (GC-rich and gene rich) and Giemsa-dark (GC-poor, gene poor) bands. Bands were assigned as GC-rich or GC-poor based on the difference in GC content between a local window of 2.5 MB and a regional window of 9.3 MB (Niimura Y, Gojobori T, 2002 supra). This chromosomal scale analysis shows that the two libraries display roughly similar but not identical levels of gene activity across the 19 autosomes and the X chromosome (Y chromosome was not evaluated). In any given chromosome, gene expression levels are not uniform and in some chromosomes, clusters signifying increased gene activity can be detected in either the TBI or sham-operated cortex.

### EXAMPLE 3

### Technical Replication Sage Results by qRT-PCR

Statistical analysis with Fisher's Exact test identified differentially-expressed genes from the two libraries. The majority of differentially-expressed genes showed under, rather than over-expression, in the TBI-library suggesting that a consequence of trauma is repressed transcription in cortical neurons during the first few hours. This picture was reinforced by qRT-PCR experiments to assess mRNA levels of traumatised and control cortical tissue.

### EXAMPLE 4

### Biological Replication of Sage Results Over a 24 hr Time Course

To confirm differential expression using multiple biological replicates (n=3 or n=5), and to detect time-related expression trends (at 2 hours, 6 hours, 12 hours, 24 hours following trauma), qRT-PCR was performed on cortical tissues after TBI, using the 2 hours sham-control as a baseline. This analysis showed that AF220209 (Nedd4-NW4BP5) was statistically significantly increased during the 24 hour period after trauma. This observation indicated those genes which are up-regulated, the increase in transcription is at high levels and detectable at 6 hours following TBI. The results of time-course for Nedd4-NW4BP5 is shown in Table 3.

### EXAMPLE 5

### Functional Classification of Differentially-Expressed Genes

GO analysis showed that down-regulated genes after the trauma were involved protein biosynthesis, endocytosis or neurotransmitter transport through binding activities such ATP binding, metal binding or protein binding while up-regulated genes were involved in ubiquitin-dependent protein activity.

TABLE 3
TIME COURSE ASSESSMENT OF mRNA LEVELS USING
BIOLOGICAL REPLICATING (p < 0.05)

| | | 2 h | 2 h | 6 h | 12 h | 24 h | |
|---|---|---|---|---|---|---|---|
| | Gene | sham | trauma | trauma | trauma | trauma | |
| Gene ID | name | (n = 5) | (n = 5) | (n = −3) | (n = 3) | (n = 3) | |
| AF220209 | Nedd4 WWdomain-bindingprotein 5 | 1 | 0.98 ± 0.14 | 2.43 ± 0.21 | 2.65 ± 0.14 | 1.99 ± 0.78 | 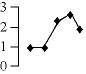 |

### EXAMPLE 6

### Genes Associates with Ubiquitination are Up-Regulated Following TBI

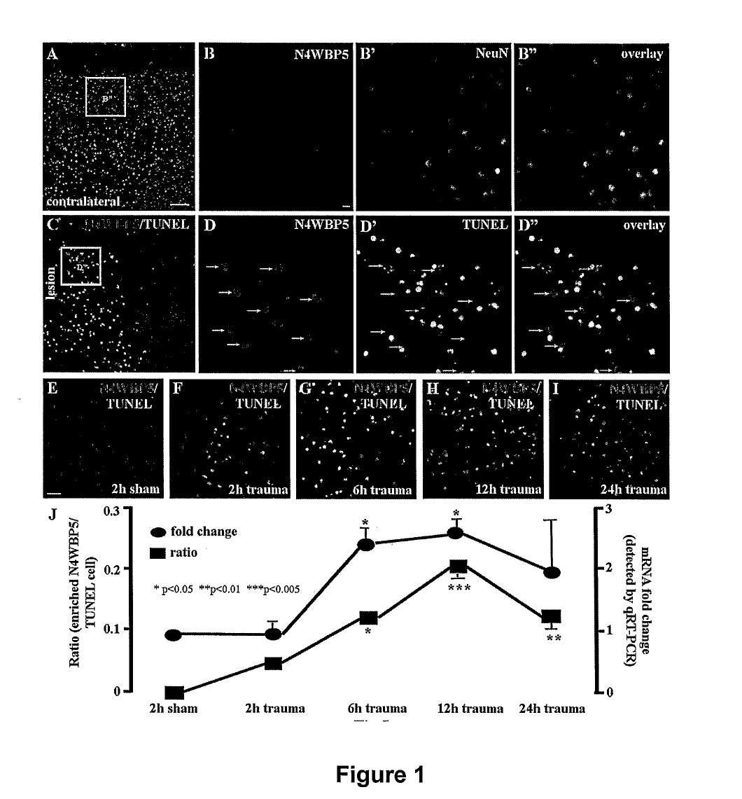

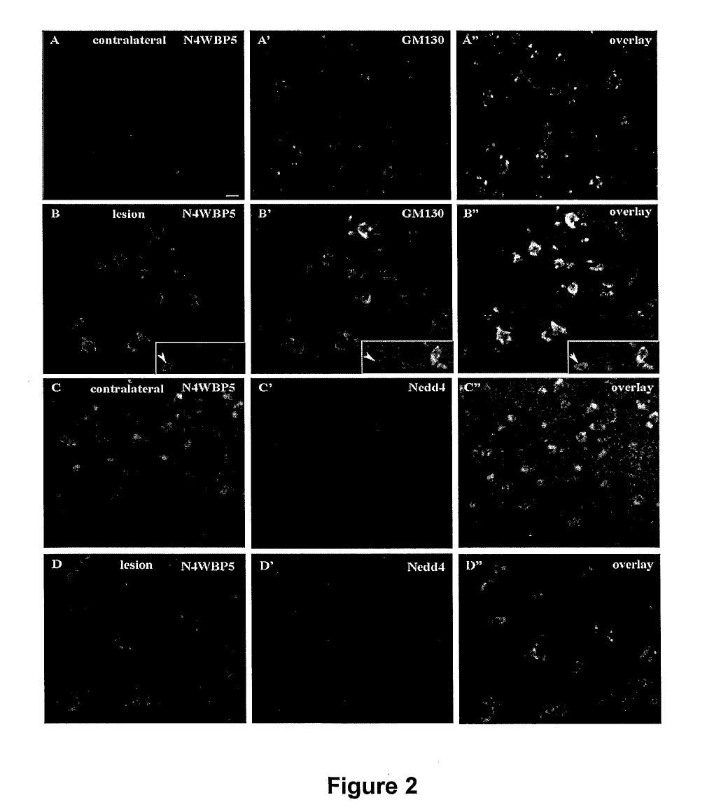

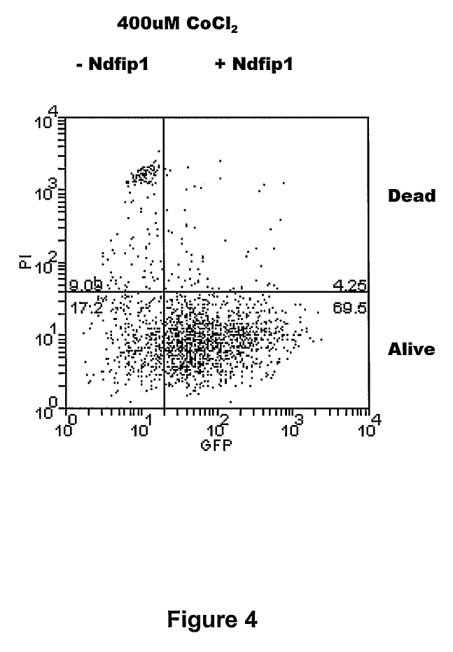

Of the up-regulated genes, Nedd4-WW domain-binding protein 5 (Ndfip1 (formally N4WBP5)) was selected for further study using antibodies raised against a GST-fusion protein (Harvey, 2002). In the hemisphere contralateral to the injury, Ndfip1 (formally N4WBP5) is normally expressed at low levels in neuronal cytoplasm (confirmed by double-staining with NeuN; FIG. 1A, B, B′, B″). In the ipsilateral hemisphere containing the trauma lesion, Ndfip1 (formally N4WBP5) protein is dramatically increased in neurons surrounding the lesion site (FIG. 1C, D, D′ D″). Importantly, none of these neurons that overexpress Ndfip1 (formally N4WBP5) is positive for TUNEL staining (FIG. 1C, D, D′ D″) suggesting that Ndfip1 (formally N4WBP5) neurons are damaged but yet to undergo apoptosis in the 2 hrs time point, or the over-expression of Ndfip1 (formally N4WBP5) is correlated with neuronal survival. To address this issue, double-staining for Ndfip1 (formally N4WBP5) and TUNEL was conducted at different time points after TBI. The results showed that Ndfip1 (formally N4WBP5) and TUNEL staining were mutually exclusive at 6 hrs, 12 hours and 24 hours after TBI (FIG. 1E-I), ruling out the possibility that staining for Ndfip1 (formally N4WBP5) at the 2 hours time-point is suggestive of subsequent cell death. Indeed this mutual exclusion for TUNEL staining was robust for all time-points examined (FIG. 1E-I). Over-expression for Ndfip1 (formally N4WBP5) around the lesion was seen only for a small number of neurons at 2 hours following TBI, this gradually increased until a maximum number of Ndfip1 (formally N4WBP5) cells was observed at the 12 hours time point followed by reduction in the 24 hour time-point (FIG. 1J). To quantify the relative numbers of Ndfip1 (formally N4WBP5)-stained neurons to TUNEL-positive cells around the lesion, we surveyed the ratio of these two cellular populations from two lesioned hemispheres at each time point. Over-expressing Ndfip1 (formally N4WBP5) neurons were never observed in sham-operated hemispheres) but the ratio of over-expressed Ndfip1 (formally N4WBP5) neurons to TUNEL-positive cells showed a statistically-significant trend (up to p<0.005; FIG. 1J) at 6 hours (0.12±0.003), 12 hours (0.20±0.02) and 24 hours (0.12±0.02) after trauma but not at 2 hours (0.05±0.008) compared to 2 hours sham (0±0). This trend appears to mirror the fold-change in mRNA levels for Ndfip1 (formally N4WBP5) detected by qRT-PCR (FIG. 4J). Collectively, these results point to Ndfip1 (formally N4WBP5) as an important marker for non-apoptotic neurons in the 24 hour period following TBI. Alternatively, Ndfip1 (formally N4WBP5) may be a marker for neurons that were initially stained for TUNEL (but negative for Ndfip1 (formally N4WBP5)) at the 2 hour or 6 hour time points, but subsequently recovered with loss of TUNEL staining and gain in Ndfip1 (formally N4WBP5) expression. Previous studies have identified Ndfip1 (formally N4WBP5) as an adaptor protein for Nedd4 in protein ubiquitination (Harvey, 2002 supra). To investigate this relationship, the cellular compartment of over-expressed Ndfip1 (formally N4WBP5) protein was localized near the lesion, and correlate its relationship to Nedd4. In the undamaged contralateral hemisphere, Ndfip1 (formally N4WBP5) staining in neurons is low-level and punctate; this staining pattern is co-extensive with the Golgi-marker GM130 suggesting the Ndfip1 (formally N4WBP5) is localized to the Golgi apparatus (FIG. 2A, A′ A″). In neurons surrounding the lesion, Ndfip1 (formally N4WBP5) staining in dramatically increased and appear ring-like around the nucleus (FIG. 2B). This staining is superimposed upon staining for the Golgi, suggesting increased Ndfip1 (formally N4WBP5) expression in localised to the Golgi (FIG. 2B, B′ B″). Occasionally, a single neuron with over-expressed Ndfip1 (formally N4WBP5) is negative for GM130 staining (FIG. 2, insets). To define its relationship to Nedd4, a ubiquitin ligase, double staining was performed using antibodies to Ndfip1 (formally N4WBP5) and Nedd4. In the contralateral hemisphere, both proteins are expressed at low levels in undamaged cortical neurons (FIG. 2C, C′ C″). Following TBI, Nedd4 expression appears to be up-regulated in the same cortical neurons showing increased expression of Ndfip1 (formally N4WBP5). Collectively, these studies point to important roles for proteins associated with ubiquitination in surviving neurons during the 24 hour period following TBI.

### EXAMPLE 7

### Over-Expression of Ndfip1 Formally N4WBP5)-Flag Protein Protects Neurons Against Death During Stress by Starvation

C57B16/J pregnant mice were killed by cervical dislocation. Mouse embryos at embryonic day 13.5 or 15.5 were removed by caesarean section and the brain was dissected. The strips of neocortex were dissected and placed in 0.1% w/v Trypsin in phosphate buffer saline (PBS) for 30 minutes at 37° C. The cells were further dissociated by passing the suspension up and down with a pipette before suspended in Dulbecco's modified Eagle Medium (DMEM, Invitrogene, Carland, Calif.) supplemented with 10% v/v foetal calf serum. The cells were plated on a poly-D-lysine (Sigma, St. Louis, Mo.) and laminin (Invitrogen) coated coverslips in a 24-well plate with 200,000 cells in 400 μl of medium per well and cultured with 5% v/v CO₂ at 37° C. overnight before the medium was replaced by Neurobasal medium (Invitrogen) supplemented with 0.5 mM L-glutamine, 1×B27 supplement (Invitrogen), 50 U/ml penicillin and 50 μg/ml streptomycin. After 6-7 days culture, the cells were washed with Neurobasal medium without antibiotics and transfected with pcDNA3-Ndfip1 (formally N4WBP5)-Flag or pcDNA3-Flag using Lipofectamine (trademark: Invitrogen) 2000 according to the manufacture's instruction (Invitrogen). In brief, 0.8 μg cDNA and 2 μl Lipofectamine (trademark: Invitrogen) 2000 were diluted in 50 μl of Opti-MEM® I Reduced Serum Medium respectively and incubated for 5 minutes before being mixed together and incubated for another 20 minutes and the complex was then added to each well and incubated for 6 hours at 37° C. with 5% v/v CO₂ The medium was then replaced with Neurobasal medium without supplements and cultured for 18 hours and fixed in 4% v/v paraformadehyde in 0.1M phosphate buffer (PB) for 2 h. After the fixative was removed with three washes by 0.1M PB, the cells were incubated in a mouse anti-Flag antibody (1:1000, Sigma) overnight. The immunoreactivity of Flag was revealed by Alexa594 conjugated goat anti-mouse IgG (1:500, Molecular Probe Inc., Eugene, Oreg.). To detect apoptotic cells, the TUNEL (TdT-mediated dUTP nick end labeling) technique was used according to the manufacture's instructions (Roche Diagnostic, Germany). The nuclei of cells were labeled with bisbenzamide (1 μg/ml, Molecular Probe).

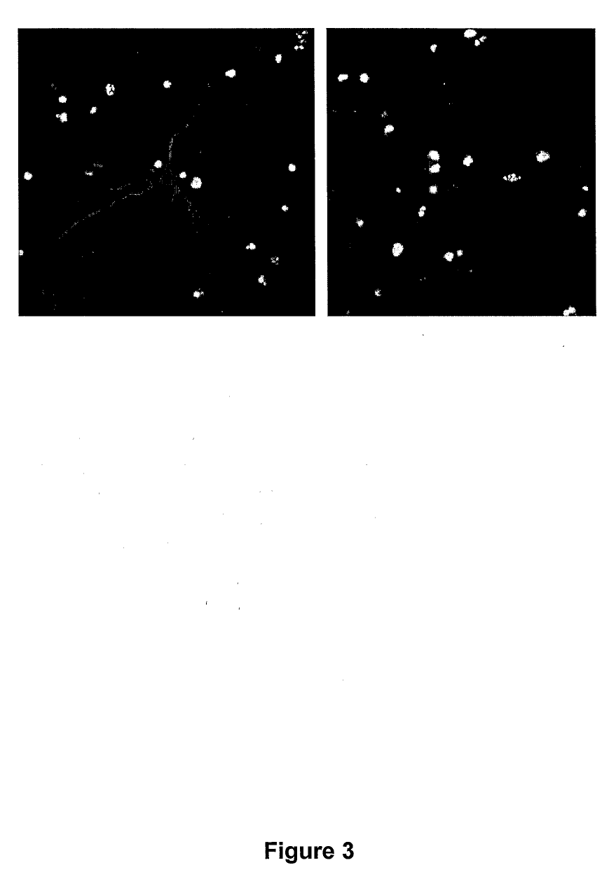

The results are shown in FIG. 3.

### EXAMPLE 8

### Ndfip1 Formally N4WBP5) Protection to Neurons During Traumatic Brain Injury

Ndfip1 (formally N4WBP5) is over-expressed in traumatized brains using a mouse model. Table 3 demonstrate that Ndfip1 (formally N4WBP5) is up-regulated by up to three fold in the injured hemisphere.

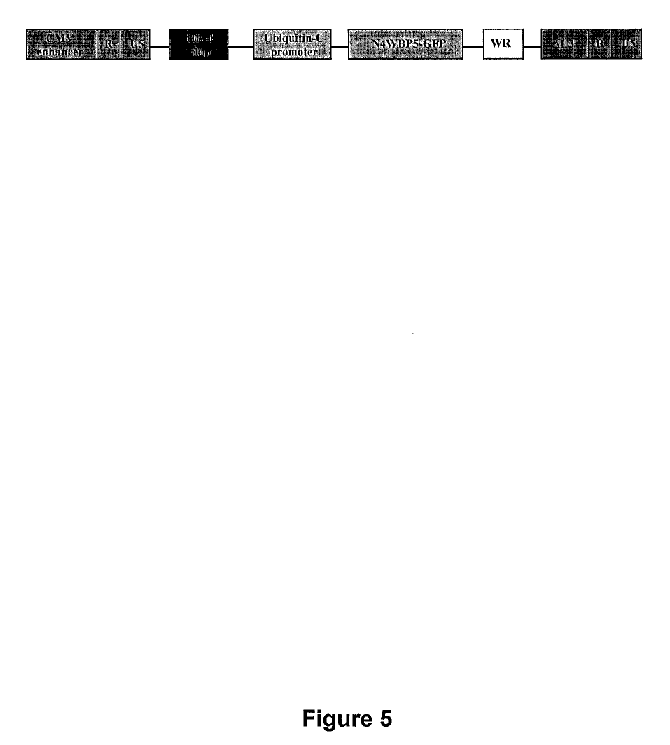

Experiments show that introduction of Ndfip1 (formally N4WBP5) in cultured embryonic cortical neurons is neuroprotective in stress by starvation (Sang et al, 2006 supra); see FIG. 5 and Table 2. Ndfip1 (formally N4WBP5) has been fused to GFP and the fusion protein delivered to cultured embryonic cortical neurons in a lentiviral vector. Infected neurons were protected from death following growth factor starvation. A third strand of evidence was obtained from studying a neural cell line, N18. Ndfip1 (formally N4WBP5) was introduced into a stable-transfected cell line and subjected the cells to stress by cobalt chloride, a known model for studying hypoxia. Using FACs sorting, it was observed that the numerical number of dying cells was significantly less in the population containing Ndfip1 (formally N4WBP5).

### EXAMPLE 9

### Ndfip1 (Formally N4WBP5) Protection During Coronary Artery Bypass Grafting (CABG)

Following CABG, many patients experience both short-term and long-term cognitive impairment. Short-term impairment occurs for up to three months after surgery whereas long-term impairment tends to occur 1 to 5 years after surgery. The aetiology is unknown but it is postulated that neuronal death (in the central nervous system) from microemboli and hypoperfusion during CABG is a major contributor. Ischemic injury to neurons can be prevented or ameliorated by up-regulation of Ndfip1 (formally N4WBP5) prior to, and during CABG. Up-regulation or Ndfip1 (formally N4WBP5) (or an agent that produces this up-regulation) is neuroprotective as a prophylactic measure administered to the patient.

This is tested using an animal model for CABG. Rats are subjected to transient ischemia by temporary occlusion of the left anterior descending artery (LAD) or circumflex artery for 5 to 20 minutes. Ndfip1 (formally N4WBP5) or mimetic agents are administered intravenously after anesthesia. The degree of myocardial damage and neuronal death is compared between animals receiving a placebo or Ndfip1 (formally N4WBP5) (or mimetic).

### EXAMPLE 10

### Ndfip1 (Formally N4WBP5) Protection During Stroke

Data are obtained showing that Ndfip1 (formally N4WBP5) is over-expressed in surviving neurons following brain ischemia induced by endothelin injection to occlude the middle cerebral artery in rats. These experiments show that neurons that up-regulate Ndfip1 (formally N4WBP5) do not stain for TUNEL, an indicator of cell death. Ndfip1 (formally N4WBP5) was seen to be over-expressed in these surviving neurons from as early as 12 hours and extending to 72 hours.

Demonstration that Ndfip1 (formally N4WBP5) is over-expressed in neurons in brain slices of rat and human tissue (obtained from postmortem, and from early pregnancy terminations) following induction of hypoxia, the predominant abnormality in cerebral ischemia is shown. Ndfip1 (formally N4WBP5) is introduced (fused to green fluorescent protein) into neurons by lentiviral infection, electroporation, or gene-gun (biolistics). Hypoxic conditions are then induced to the brain slice and the expression and movement of Ndfip1 (formally N4WBP5)/GFP fusion protein monitored. Neuroprotection to neurons over-expressing the fusion protein is expected.

### EXAMPLE 11

### Ndfip1 (Formally N4 WBP5) Protection During Hypoxia in Preterm Infants

Preterm children who develop severe chronic lung disease (bronchopulmonary dysplasia) may be developmentally compromised by exposure to hypoxic episodes. Chronic hypoxia affects the developing brain and may contribute to increased neuronal death during the critical period of synaptogenesis and pruning. In humans, this could lead to long-term impairments in visual-motor, gross and fine motor, articulation, reading, mathematics, spatial memory and attention skills.

Ndfip1 (formally N4WBP5) is proposed to be protective against neuronal death from hypoxic episodes in preterm infants and children if Ndfip1 (formally N4WBP5) (or mimetic) is introduced into neurons. Evidence that Ndfip1 (formally N4WBP5) is protective in neuronal cell line N-18 is obtained. Hypoxia is induced using cobalt chloride, a known chemical for mimicking hypoxia. In N18 cells containing introduced Ndfip1 (formally N4WBP5), there was a statistically increased neuroprotection from death, as measured by propidium iodide stain and counted by FACs sorting (FIG. 4).

### EXAMPLE 12

### Ndfip1 (Formally N4 WBP5) Protection in Hypoxic Conditions of the Eye

The retina, containing photoreceptors, is very sensitive to oxygen levels. In diseases that cause low levels of oxygen in the blood (heart, lung, diabetes) can cause retinal hypoxia. This can lead to retinal diseases such as von Hippel-Lindau, retinitis pigmentosa, proliferative diabetic retinopathy, reintopathy of prematurity and glaucoma. Based on its action in the brain, it is proposed that increased Ndfip1 (formally N4WBP5) can protein neurons in the retina, particularly the rod and con photoreceptors from injury and death in these conditions.

### EXAMPLE 13

### Ndfip1 (Formally N4WBP5) Protection of Healthy Neurons During Tumor Irradiation

During ionizing irradiation of the brain to treat brain tumors in young children, there is collateral damage causing death of normal neurons. It is proposed that over-expression of Ndfip1 (formally N4WBP5) in these situations increases the survival of irradiated neurons but not part of the tumor.

### EXAMPLE 14

### Human Studies of Ndfip1 (Formally N4WBP5)

To investigate the neuroprotective role of Ndfip1 (formally N4WBP5) in humans, normal and abnormal expression of Ndfip1 (formally N4WBP5) in human neurons in adult brains following traumatic brain injury (TBI) is monitored. The level and pattern of expression of Ndfip1 (formally N4WBP5) is mapped in human brain tissue. Ndfip1 (formally N4WBP5) expression is examined in cortical neurons, comparing damaged and intact hemispheres. The relative levels of Ndfip1 (formally N4WBP5) expression is plotted at different time-points post-injury, and related to the severity of the lesion. In addition to immunocytochemistry, biochemical analysis of Ndfip1 (formally N4WBP5) (mRNA and protein) expression is conducted using Northern (or real-time PCR) and Western blotting. This study is able to correlate Ndfip1 (formally N4WBP5) expression with TBI in human brains and provide a foundation for intervention strategies to up-regulate Ndfip1 (formally N4WBP5) in TBI. Ndfip1 (formally N4WBP5) levels are manipulated in cultured human neurons. These neurons are cultured and the Ndfip1 (formally N4WBP5) gene introduced into them using transfection or lentiviral vectors or electroporation.

### EXAMPLE 15

### Mechanisms and Targets of Neuroprotection by N4WBP5

### Mechanisms

In order to ascertain how Ndfip1 (formally N4WBP5) is able to confer neuroprotection following trauma, in vitro work is designed to understand the biochemistry behind this protection. This is important for two reasons. First, the biochemical and molecular information will underpin in vivo studies on animal models and also provide a framework for designing and if necessary, for modifying the in vivo delivery of Ndfip1 (formally N4WBP5) into the traumatized brain. Second, understanding the biochemical pathways of Ndfip1 (formally N4WBP5) protection leads to the identification of new targets for therapeutic intervention. Furthermore, if Ndfip1 (formally N4WBP5) action is understood, then drugs can be designed to evoke this function.

In vitro studies investigate a number of questions. First, to define the nature of the death-inducing stimuli that is protected by Ndfip1 (formally N4WBP5) and to determine if Ndfip1 (formally N4WBP5)-mediated protection is specific to neurons or to other cell types. This has ramifications for treating traumatic injury to non-neuronal tissues (e.g. heart muscle). An important first question will be whether or not Ndfip1 (formally N4WBP5) can confer neuroprotection against both intrinsic death signals (cell stress) or extrinsic signals (death ligands). To answer this, neural and non-neural cell lines are used for the studies (neuroblastoma, fibroblast, epithelial, leukemia and lymphoma cell lines). Ndfip1 (formally N4WBP5) expression is used in these cell lines and then exposed to various death stimuli that initiate cell death using either extrinsic signals (e.g. death ligands) or intrinsic signals (cell stress).

The above experiments use a reliable assay that quantifies the level of protection. Following transfection of Ndfip1 (formally N4WBP5) with either plasmid vector (N4WBP5-GFP) or lentivirus expression vectors (coupled to GFP), cell lines are examined for infection/transfection efficiency, and sorted by flow cytometry for GFP expression. Cells will be treated with an apoptosis-inducing agent and cell lysates are prepared for testing for caspase activity using fluorogenic substrates. Cell death are quantitated directly by counting the number of apoptotic cells using nuclear condensation as a marker of apoptotic morphology, as visualized using Hoechst or DAPI staining. Biochemical markers of the apoptotic pathway (Bax translocation and cytochrome c release into the cytosol) are assessed by immunofluorescence and cell fractionation.

Data suggest that Ndfip1 (formally N4WBP5) protection is mediated via the NFκB and MAPK pathways. This is pursued by employing inhibitors of NFκB and MAPK pathways on cell lines expressing N4WBP5, and to test whether blocking either of these pathways abrogates the neuroprotective effects of Ndfip1 (formally N4WBP5). These pathways are directly linked to the apoptotic pathway mediated by BclX, cIAPs and Bim. The expression of these genes is monitored via PCR and immunoblotting.

### Targets

Ndfip1 (formally N4WBP5) is proposed to act as a bridge between Nedd4 family of E3 ubiquitin ligases and their targets, leading to ubiquitination of damaged proteins following stress in TBI. Several Nedd4 family members associate with the PY motifs of the Ndfip1 (formally N4WBP5). It is predicted that the cytosolic N-terminus region of Ndfip1 (formally N4WBP5) is most likely to interact with other components of the ubiquitination/trafficking machinery, although binding of specific proteins through the small loop region between TM2 and TM3, and to the C-termini cannot be ruled out. To cover all possibilities, the use of both full-length protein and the N-terminal regions of the Ndfip1 (formally N4WBP5) in co-immunoprecipitation experiments is used.

### Identification of N4WBP5-Interacting Proteins

Immunoprecipitation (IP) is used to pull down the Ndfip1 (formally N4WBP5) and associated proteins and identify interacting proteins using peptide mass fingerprinting. The data show that the IPs from cells transfected with FLAG-tagged N4WBPs are much cleaner than those using polyclonal antibodies and endogenous proteins. Therefore, N18 cells are used which are transfected with expression vectors carrying the FLAG-tagged full length Ndfip1 (formally N4WBP5) or the N-terminal 115 amino acid (aa) region of the Ndfip1 (formally N4WBP5) and, following 48 hours, IP proteins with FLAG antibody-coupled agarose. A well established protocol for these experiments is used together with generated data using ³⁵S-Met labeled cells that clearly indicate the presence of a number of binding partners for these proteins in IPs. IP proteins are resolved by SDS-PAGE and visualized with Sypro Ruby. Individual bands are excised and subjected to in-gel digestion with trypsin. Peptides are extracted and resolved by capillary (75 μm) reverse-phase chromatography into Q-TOF2 MS equipped with a nanospray ion-source. Automated collision-induced dissociation is performed and the data (peptide masses and daughter ions) used to interrogate the NCBI-NR protein database using Waters ProteinLynx software.

Known proteins with established functions in the NFkB or MAPK pathways assist in predicting the precise function of the Ndfip1 (formally N4WBP5) in cell survival pathways. Basic characterization of all potentially interesting binding proteins (prioritised based on their known/predicted functions) is carried out. Interactions with Ndfip1 (formally N4WBP5) is confirmed by IP experiments in which the proteins are ectopically expressed in neuronal cells. The identified proteins are also used as substrates in ubiquination assays with the E3s Nedd4 or Nedd4-2. These assays are well established in the art. Other studies include assessment of localization, and turnover of the identified proteins in cells with ectopically expressed or reduced/ablated expression of Ndfip1 (formally N4WBP5). The role in cell survival of neuronal cells is further assessed by siRNA-mediated depletion using in vitro assays and in animal models.

Outcome (1):
(1) Produce cell lines stably expressing Ndfip1 (formally N4WBP5).
(2) Identify the biochemical basis of neuroprotection by Ndfip1 (formally N4WBP5), with respect to cell death pathways.
(3) Clarify the involvement of Ndfip1 (formally N4WBP5) in NFκB and MAPK pathways.
(4) Identify proteins that bind to Ndfip1 (formally N4WBP5) in a stressed cellular environment
(5) Verify molecular interactions between Ndfip1 (formally N4WBP5) and Nedd5 in the ubiquitination and disposal of damaged proteins

Using information gained in outcome (1), the studies are extended into animal models to test its neuroprotective function. Closed head injury model is used to assess whether or not increasing or decreasing expression of Ndfip1 (formally N4WBP5) in stressed neurons has the ability to modify the number of surviving neurons (versus apoptotic neurons) around the lesion. Such immunohistological studies are complemented by monitoring the neurological recovery of the mice with a range of behavioral testing regimes such as rotor rod and locomotor cell tests to test for rearing and motor behavior; and Morris Water Maze and Barnes Radial Maze to monitor cognitive and sensory recovery. The aim is to increase the expression of Ndfip1 (formally N4WBP5) in mouse brains using a variety of routes listed below.

### (1) Lentiviral Infection Expressing N4WBP5-GFP in the Lesion.

The trauma lesion is directly infected with lentiviral particles. An expression construct is made and tested that allows identification of the infected neurons via a C-terminal GFP fusion to the Ndfip1 (formally N4WBP5) protein (FIG. 5). Pilot investigations using this construct demonstrate specific expression of Ndfip1 (formally N4WBP5)-GFP in N18 and SN56 cells which is consistent with the localization of the native Ndfip1 (formally N4WBP5) protein to the Golgi apparatus. It is planned to infect neurons around the trauma site immediately after TBI using replication-incompetent lentiviral particles (with or without pseudotyping with stomatotitis virus VSV-G protein). GFP-positive cells around the lesion are assayed at various timepoints for evidence of rescue and corelated with TUNEL co-staining to ascertain if they are apoptotic or not. It is expected to observe an inverse relationship between numbers of GFP-positive neurons with TUNEL.

### (2) Over-Expression of N4WBP5 in Transgenic Mice/KO Mice

A transgenic mouse over-expressing Ndfip1 (formally N4WBP5) under the ubiquitin C-promoter is made. This construct is capable of infecting fertilized eggs following introduction of the viral particles into the zona and GFP reporter expression in the brain. High rates of infection and even higher rates of transgenesis (80%) above conventional methods that use pronuclei injection (typically 5%) are expected to be seen. Expression is monitored by GFP fluorescence and staining using Ndfip1 (formally N4WBP5) antibodies. Once transgenic lines expressing Ndfip1 (formally N4WBP5) are obtained, then neuroprotection tests are conducted on their cortical neurons. This includes cellular and behavioral analysis following TBI to look for evidence of increased survival and decreased apoptosis. Functional and behavioural testing following TBI, compared to control littermates, is also performed. As a counterpoint, a knock-out mouse lacking Ndfip1 (formally N4WBP5) is generated.

### (3) TAT-Mediated Delivery of N4WBP5

TAT is an 11-aa sequence developed from HIV-1 Tat protein capable of delivering full-length proteins into cells with biological activity. It is capable of delivering recombinant proteins (up to 1000 aa) across the cell membrane and, among other applications, has been shown capable of delivering anti-apoptotic proteins such as Bcl-xL into the brain following ischaemic injury, reducing apoptosis and infarct volume. Fusion of the Ndfip1 (formally N4WBP5) to the pTAT-hemagglutinin (HA) vector generates TAT-N4WBP5 in bacteria followed by 6× histidine purification. Following purification in Ni²⁺-NTA-agarose affinity columns and FPLC, the fusion protein will be tested for its neuroprotective effects in primary cortical neurons before introduction into the brains of mice at various timepoints, before and after TBI. TAT-BCL-xL has been shown to cross the blood-brain barrier following systemic administration. TAT-N4WBP5 is introduced sytematically (i.p. or i.v.) before and after TBI induction. Neurons taking up TAT-Ndfip1 (formally N4WBP5) are identified using antibodies against HA and protein levels are measured in immunoblots. The most effective treatment regime (dose and timing of administration) is titrated using cell-based scoring of apoptosis in treated brains coupled to behavioral testing.

In conclusion, it is expected that the above experiments demonstrate a neuroprotective effect of Ndfip1 (formally N4WBP5). The experiments are extended into humans. The human Ndfip1 (formally N4WBP5) has been cloned and it is tested for Ndfip1 (formally N4WBP5) neuroprotective effect in human brain cells using cultured cortical neurons.

Ascertain that N4WBP5 is neuroprotective in animal models of TBI and correlate the degree of protection with the cellular and behavioural attributes of recovering mice.

Outcome (2):
(1) Produce lentiviral constructs expressing functional Ndfip1 (formally N4WBP5)-GFP protein.
(2) Successful rescue of dying neurons by lentiviral expression of Ndfip1 (formally N4WBP5) in TBI lesions.
(3) Successful generation of transgenic mice expressing Ndfip1 (formally N4WBP5) using lentiviral vectors.
(4) Successful generation of targeting vector and production of knock-out mice for Ndfip1 (formally N4WBP5).
(5) Demonstrate neuroprotection by Ndfip1 (formally N4WBP5)-expressing transgenic mice.
(6) Produce TAT-Ndfip1 (formally N4WBP5) vector for systemic delivery of Ndfip1 (formally N4WBP5) following TBI.

The outcomes include a demonstration of neuroprotection following administration of TAT-Ndfip1 (formally N4WBP5) into rodent models.

### EXAMPLE 16

### Transport of Ndfip1

Data was obtained to suggest that cells secrete Ndfip1 into the extracellular space. In culture experiments, secreted Ndfip1 is detected in immunoprecipitation experiments using culture supernatant. This indicates an ability of Ndfip1 to be secreted into the environment to rescue other cells The implication is that introduction of Ndfip1 into the site of injury in the brain directly give succour to stressed neurons by penetration into neurons. Experiments show that Ndfip1-Flag fusion protein is secreted into culture supernatant following transfection in 293T cells. Following immunoprecipitation of supernatant with anti-Ndfip1 or anti-Flag antibodies, a positive band is immunoreactive with antibodies to Ndfip1/Flag.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Adams et al, Cancer Res 53:4026-4034, 1993
Altschul et al, Nucl Acids Res 25:3389-3402, 1997
Alving et al, Immunol Rev 145:5-31, 1995
Atherton and Shephard, Synthetic Vaccines, Chapter 9, published by Blackwell Scientific Publications
Ausubel et al, Current Protocols in Molecular Biology, John Wiley & Sons Inc, Chapter 15, 1994-1998
Ausubel (Ed), Current Protocols in Molecular Biology, 5^{th} Edition, John Wiley & Sons, Inc, NY, 2002
Bandyopadhyay and Temin, Mol Cell Biol 4:749-754, 1984
Berglund et al, Biotechnology 11:916-920, 1993
Berkner et al, BioTechniques 6:616-629, 1988
Berkner, Curr Top Microbiol Immunol 158:39-66, 1992
Bradely et al., Nature 309:255-258, 1984
Breakefield and Geller, Mol Neurobiol 1:339-371, 1987
Buchschacher and Panganiban, J Virol 66:2731-2739, 1982
Capecchi et al, Trends Genet. 5:70-76, 1989
Capecchi et al., Science 244:1288-1292, 1989
Chothia et al, J Mol Biol 196:901-917, 1987
Coligan et al., Current Protocols in Immunology, John Wiley & Sons, Inc., 1991-1997
Cumber et al, J Immunol 149:120-126, 1992
Davies and Riechmann, FEBS Lett 339:285-290, 1994
Deutscher (Ed), Methods Enzymol 182:147-238, 1990
Di Giovanni et al, Proc Natl Acad Sci USA 102:8333-8338, 2005
European Patent Publication No. 0 239 400
Evans et al., Nature 292:154-156, 1981
Faden A I, Curr Opin Neurol 15:707-712, 2002
Fink et al, Hum Gene Ther 3:1-19, 1992
Fink et al, Ann Rev Neurosci 19:265-287, 1996
Fix, Pharm Res 13:1760-1764, 1996
Freese et al, Biochem Pharmaco. 40:2189-2199, 1990
Glockshuber et al, Biochem 29:1363-1367, 1990
Gossler et al., Proc Natl Acad Sci U.S. 83:9065-9069, 1986
Gorziglia and Kapikian, J Virol 66:4407-4412, 1992
Hamers-Casterman et al, Nature 363:446-448, 1993
Helseth et al, J Virol 64:2416-2420, 1990
Hodgson, BioTechnology 9:19-21, 1991
Hogan et al., Manipulating the Mouse Embryo: A Laboratory Manual, Cold Spring Harbor Laboratory Press, Cold Spring Harbor N.Y., 1986
Johnson et al, J Virol 66:2952-2965, 1992
Johnson et al., Peptide Turn Mimetics in Biotechnology and Pharmacy, Pezzuto et al., Eds., Chapman and Hall, New York, 1993
Jones et al, Nature 321:522-525, 1986
Kennet et al. ((eds) Monoclonal Antibodies and Hybridomas: A New Dimension in Biological Analyses, pp. 376-384, Plenum Press, New York, 1980
Keyvani et al, J Neruopathol Exp Neurol 63:598-609, 2004
Kobori, Brain Res Mol Brain Res 104:148-158, 2002
Köhler and Milstein, Nature 256:495-499, 1975; Köhler and Milstein, Eur J Immunol 6:511-519, 1976
Kozbor et al, Methods in Enzymology 121:140-167, 1986
Krebber et al, J Immunol Methods 201:35-55, 1997
Ku and Schutz, Proc Natl Acad Sci USA 92:6552-6556, 1995
Langer, Science 249:1527-1533, 1990
Liu et al, Proc Natl Acad Sci USA 84:3439-3443, 1987
Madzak et al, J Gen Virol 73:1533-1536, 1992
Mann and Baltimore, J Virol 54:401-407, 1985
Mansour et al., Nature 336:348-352, 1988
Margolskee, Curr Top Microbiol Immunol 158:67-95, 1992
Miller et al, Mol Cell Biol 5:431-437, 1985
Miller et al, J Virol 62:4337-4345, 1988
Miller, Curr Top Microbiol Immunol 158:1-24, 1992
Misra et al, J Pharm Sci 6:252-273, 2003
Moss, Curr Top Microbiol Immunol 158:5-38, 1992
Moss, Proc Natl Acad Sci USA 93:11341-11348, 1996
Munder et al. (Appl Microbiol Biotechnol 52:311-320, 1999
Muzyczka, Curr Top Microbiol Immunol 158:97-129, 1992
Naldini et al, Science 272:263-267, 1996
Natale et al, J Neurotrauma 20:907-927, 2003
Niimura Y, Gojobori T, Proc Natl Acad Sci USA 99:797-802, 2002
Ohi et al, Gene 89:279-282, 1990
Page et al, J Virol 64:5270-5276, 1990
Patton, Nat Biotech 16:141-143, 1998
Petropoulos et al, J Virol 66:3391-3397, 1992
Pickert, Transgenic Animal Technology: A Laboratory Handbook. Academic Press, San Diego, Calif., 1994
Plückthun et al (In Antibody engineering: A practical approach 203-252, 1996
Putney and Burke, Nat Biotech 16:153-157, 1998
Quantin et al, Proc Natl Acad Sci USA 89:2581-2584, 1992
Rall et al, Neruopathol Appl Nerobiol 29:118-131, 2003
Rao et al, J Neruotrauma 16:865-877, 1999
Reiter et al, J Biol Chem 269:18327-18331, 1994
Reiter et al, Biochem 33:5451-5459, 1994
Reiter et al, Cancer Res 54:2714-2718, 1994
Remington's Pharmaceutical Sciences, 18^{th} Edition, Mack Publishing Company, Easton, Pa., 1990
Remington's; Akimaru et al, Cytokines Mol Ther 1:197-210, 1995
Remington's; Egleton and Davis, Peptides 18:1431-1439, 1997
Riechmann et al, Nature 332:323-327, 1988
Robertson (Ed), Teratocarcinomas and Embryonic Stem Cells: A Practical Approach. IRL Press, Washington D.C., 1987
Robertson et al., Nature 322:445-448, 1986
Rosenfeld et al, Cell 68:143-155, 1992
Russell and Hirata, Nat Genetics 18:323-328, 1998
Samanen et al, J Pharm Pharmacol 48:119-135, 1996
Sambrook and Russell, Molecular Cloning: A Laboratory Manual, 3^{rd} Edition, Cold Spring Harbor Laboratory Press, Cold Spring Harbor, N.Y., 2001
Sang et al, The Journal of Neuroscience 26(27):7234-7244, 2006
Sayani and Chien, Crit. Rev Ther Drug Carrier Syst 13:85-184, 1996
Schneider et al, Nat Genetics 18:180-183, 1998
Shimada et al, J Clin Invest 88:1043-1047, 1991
Shulman et al, Nature 276:269-270, 1978; Volk et al, J Virol 42:220-227, 1982
Sorge et al, Mol Cell Biol 4:1730-1737, 1984
Stratford-Perricaudet et al, Hum Gene Ther 1:241-256, 1990
Suntres and Shek, J Pharm Pharmacol 46:23-28, 1994
Szoka and Papahadjopoulos, Ann Rev Biophys Bioeng 9:467-508, 1980
Toyama et al., “Monoclonal Antibody, Experiment Manual”, published by Kodansha Scientific, 1987
Trowbridge, J Exp Med 148:313-323, 1978
U.S. Pat. No. 4,235,871
U.S. Pat. No. 4,501,728
U.S. Pat. No. 4,837,028.
U.S. Pat. No. 5,091,513
U.S. Pat. No. 5,283,185
U.S. Pat. No. 5,279,833
U.S. Pat. No. 5,391,377
U.S. Pat. No. 6,056,957
U.S. Pat. No. 6,096,716
U.S. Pat. No. 6,110,490
U.S. Pat. No. 6,180,370
U.S. Pat. No. 6,180,377
Verhoeyen et al, Science 239:1534-1536, 1988; Riechmann et al, Nature 332:323-327, 1988
Vutla et al, J Pharm Sci 85:5-8, 1996
Ward et al, Nature 341:544-546, 1989
Webber et al, Mol Immunol 32:249-258, 1995
Wells, Methods Enzymol 202:2699-2705, 1991
Wilkinson et al, Nucleic Acids Res 20:233-2239, 1992
Winter and Milstein, Nature 349:293-299, 1991
Woodle et al, Pharm Res 9:260-265, 1992
Yoshiya et al, J Neurotrauma 20:1147-1162, 2003
Young et al, Nat Biotechnol 16:946-950, 1998
Zalipsky et al, Bioconjug Chem 6:705-708, 1995

1. A method for the treatment or prophylaxis of a disease or condition in a subject said method comprising up-regulating expression of Ndfip1 or otherwise increasing levels of Ndfip1 in cells of said subject.

2. The method of claim 1 wherein the subject is a human.

3. The method of claim 2 wherein the disease or condition is trauma to the brain.

4. The method of claim 2 wherein the disease or condition is coronary artery bypass grafting (CABG).

5. The method of claim 2 wherein the disease or condition is hypoxia in preterm infants.

6. The method of claims 2 wherein the disease or condition is hypoxic conditions of the eye.

7. The method of claim 2 wherein the disease or condition is tumor irradiation or chemotherapy.

8. The method of claim 3 wherein the trauma of the brain is caused by acute neurological disease or traumatic injury.

9. The method of claim 8 wherein the neurological disease or traumatic injury is a severe head injury, trauma-induced paralysis, infection or starvation.

10. A therapeutic or prophylactic composition comprising an agent which elevates levels of Ndfip1 or Ndfip1-Nedd4 interaction and one or more pharmaceutical carriers, diluents and/or excipients.

11. The therapeutic or prophylactic composition of claim 10 wherein the agent is a nucleic acid molecule capable of being expressed and encoding Ndfip1.

12. The therapeutic or prophylactic composition of claim 11 wherein the nucleic acid molecule is a viral construct or present in a virosome or liposoine.

13. The therapeutic or prophylactic composition of claim 10 wherein the agent is Ndfip1 protein.

14. The therapeutic or prophylactic composition of claim 13 wherein the Ndfip1 protein is a virosome or liposome.

15. The therapeutic or prophylactic composition of claim 10 wherein the agent is a small molecule mimetic of Ndfip1.

16. A method for determining the presence of potential disease or condition in a subject said method comprising determining the levels of Ndfip1 or Ndfip1-expression or Ndfip1-Nedd4 interaction wherein an increase in levels or expression is indicative of disease or condition.

17. The method of claim 16 wherein the subject is a human.

18. The method of claim 17 wherein the disease or condition is trauma to the brain.

19. The method of claim 17 wherein the disease or condition is coronary artery bypass grafting (CABG).

20. The method of claim 17 wherein the disease or condition is hypoxia in preterm infants.

21. The method of claims 17 wherein the disease or condition is hypoxic conditions of the eye.

22. The method of claim 17 wherein the disease or condition is tumor irradiation or chemotherapy.

23. The method of claim 18 wherein the trauma of the brain is caused by acute neurological disease or traumatic injury.

24. The method of claim 23 wherein the neurological disease or traumatic injury is a severe head injury, trauma-induced paralysis, infection or starvation.

25-33. (canceled)


SEQUENCE LISTING (4 sequences)

SEQ ID NO: 1 - DNA, 1837 bp, Homo sapiens
ccaaggcgcgtccccctcggcctcccagcgctcccaagccgcagcggccgcgccccttcagctagctcgctcgctcgctctgcttccctgctgccggctgcgccatggcgttggcgttggcggcgctggcggcggtcgagccggcctgcggcagccggtaccagcagttgcagaatgaagaagagtctggagaacctgaacaggctgcaggtgatgctcctccaccttacagcagcatttctgcagagagcgcagcatattttgactacaaggatgagtctgggtttccaaagcccccatcttacaatgtagctacaacactgcccagttatgatgaagcggagaggaccaaggctgaagctactatccctttggttcctgggagagatgaggattttgtgggtcgggatgattttgatgatgctgaccagctgaggataggaaatgatgggattttcatgttaacttttttcatggcattcctctttaactggattgggtttttcctgtctttttgcctgaccacttcagctgcaggaaggtatggggccatttcaggatttggtctctctctaattaaatggatcctgattgtcaggttttccacctatttccctggatattttgatggtcagtactggctctggtgggtgttccttgttttaggctttctcctgtttctcagaggatttatcaattatgcaaaagttcggaagatgccagaaactttctcaaatctccccaggaccagagttctctttatttattaaagatgttttctggcaaaggccttcctgcatttatgaattctctctcaagaagcaagagaacacctgcaggaagtgaatcaagatgcagaacacagaggaataatcacctgctttaaaaaaataaagtactgttgaaaagatcatttctctctatttgttcctaggtgtaaaattttaatagttaatgcagaattctgtaatcattgaatcattagtggttaatgtttgaaaaagctcttgcaatcaagtctgtgatgtattaataatgccttatatattgtttgtagtcattttaagtagcatgagccatgtccctgtagtcggtagggggcagtcttgctttattcatcctccatctcaaaatgaacttggaattaaatattgtaagatatgtataatgctggccattttaaaggggttttctcaaaagttaaacttttgttatgactgtgtttttgcacataatccatatttgctgttcaagttaatctagaaatttattcaattctgtatgaacacctggaagcaaaatcatagtgcaaaaatacatttaaggtgtggtcaaaaataagtctttaattggtaaataataagcattaattttttatagcctgtattcacaattctgcggtaccttattgtacctaagggattctaaaggtgttgtcactgtataaaacagaaagcactaggatacaaatgaagcttaattactaaaatgtaattcttgacactctttctataattagcgttcttcacccccacccccacccccaccccccttattttccttttgtctcctggtgattaggccaaagtctgggagtaaggagaggattaggtacttaggagcaaagaaagaagtagcttggaacttttgagatgatccctaacatactgtactacttgcttttacaatgtgttagcagaaaccagtgggttataatgtagaatgatgtgctttctgcccaagtggtaattcatcttggtttgctatgttaaaactgtaaatacaacagaacattaataaatatctcttgtgtagcaaaaaaaaaaaaaaaaaaaaa

SEQ ID NO: 2 - protein, 221 aa, Homo sapiens
MALALAALAAVEPACGSRYQQLQNEEESGEPEQAAGDAPPPYSSISAESAAYFDYKDESGFPKPPSYNVATTLPSYDEAERTKAEATIPLVPGRDEDFVGRDDFDDADQLRIGNDGIFMLTFFMAFLFNWIGFFLSFCLTTSAAGRYGAISGFGLSLIKWILIVRFSTYFPGYFDGQYWLWWVFLVLGFLLFLRGFINYAKVRKMPETFSNLPRTRVLFIY

SEQ ID NO: 3 - DNA, 5494 bp, Mus musculus
cacgcttgctccaacccagccccccaagcctcgcgcgcagccgcagagcgggtcccgccccttctcgccgcgcgcccctgcaccccagcgcgctcccggagtgggcctcctccgccgcagcgacgcctcctcctcctcttcctctctccttctctctcggtctctcgctctctctcgccgctgcagcctagtaggggcgcttcgtccagcatgagctcggacatggcagccgacgagtcggaggccccagtactctcggaggacgaggtatgggagttttgcctggataagacagaagatggtggcggatcccccggaagtgatgttacagatacttgtgagcctccatgtggatgctgggagttgaatccgaattccctggaagaggagcacgtgctgttcactgctgatccgtacctggagctccacaacgatgacacacgagttgtgagagtgaaggttatagctggcataggcctggccaagaaagacatcttgggagccagtgatccttacgtaagagtgacattgtatgacccgatgagtggaatccttaccagcgtgcagacaaaaactatcaaaaagtctttgaatccaaaatggaatgaagaaatactgttcagggtccttccacagcgacaccgcattcttttcgaagtgtttgatgaaaatcgtttgacaagagatgatttcctaggtcaagtggatgtccctctctatcctttaccgactgaaaacccaagaatggagagaccatatacatttaaggattttgttcttcatccaagaagtcacaaatcaagagttaaaggttatctgagattaaaaatgacttatttacctaaaaatggctcagaagatgaaaatgcagaccaggctgaggagttagagcctggctgggttgttttggaccaaccagatgctgccactcatttgccgcatccaccagaaccctctcccctacctccaggatgggaagagaggcaggatgtccttggaaggacctactacgtaaaccatgaatctagaagaacacagtggaaaaggccaagccctgacgatgacctcacggatgaagacaatgatgatatgcagctgcaagcgcagcgagcattcaccaccaggcggcagatatcggaggatgtggatggccctgacaaccgggagtcccctgagaattgggaaatcgtacgagaagatgaaaacaccgagtatagtggtcaggctgtccagtcacctccatcgggtcacattgatgtgcagactcaccttgcagaagagtttaataccagacttgctgtgtgtggaaatccagccaccagccagccggttaccagctcaaatcattccagcagaggaggcagcttgcagacctgtatctttgaggaacagcctacacttcctgtgcttttgcctacttcatctggattgccaccaggttgggaagaaaaacaagatgacagaggaagatcatactatgtagaccacaactctaaaaccaccacatggtccaagcccaccatgcaggatgatccaagatcgaaaatccctgctcatctgagaggaaagactgactccaatgacctgggacccttacctccaggctgggaagaaagaacccacacagatgggcgagtcttcttcataaaccacaatataaagaagacccagtgggaagatcctcgcctgcagaacgtggcaatcactggaccagcagtgccctactccagagattacaagagaaagtacgagttcttcagaaggaagctcaagaagcagactgacattccaaacaaatttgaaatgaagcttcgccgcgcaaacattctggaggattcttaccggaggattatgggtgtgaagagagctgacttgctcaaggccagactctggattgagtttgatggtgaaaagggccttgactatggaggggttgccagagagtggttcttcctcatctcgaaggaaatgttcaacccttactacggcctgtttgaatattctgctacggataattacaccctacagataaatcctaactcgggcttgtgtaatgaagatcacctctcatacttcaagttcattggccgtgtggctgggatggcagtttatcatggcaagctgttggatgggtttttcatccgtccgttttacaagatgatgcttcagaaactgataacactgcacgacatggagtccgtggatagtgaatattacagttctctgcgatggattcttgaaaatgacccgacggagctggacctgagatttatcatagatgaagaactttttggacagacacatcagcacgaactgaaaaccggaggatcagagattgttgtcaccaataagaacaaaaaggagtatatctaccttgtaatacaatggcgatttgtgaaccgtatccagaagcaaatggcagcttttaaagagggattttttgaactgataccacaggatctcatcaagatatttgatgaaaatgagctagagcttctcatgtgtggtctgggagatgtggatgtgaacgactggcgggaacacacaaaatacaaaaatggctacagcatgaaccaccaggtcatccactggttctggaaggctgtttggatgatggattcggaaaaaagaatacgcttacttcagtttgtcactggcacatcccgtgtgccgatgaatgggtttgctgaactctatggctcgaatggaccacaatccttcacagtggaacaatggggcacccctgataagctgccaagagcacacacctgcttcaatcgcctggacctgccaccctacgaatcctttgacgaactctgggataaacttcagatggcaattgagaacacacagggctttgatggcgttgattagattacaaaaaaaaacaagctgtggtgtcttgtacaccatagtttctaagcaaaagcaaaaacaaaatcacattttaacattttccagatagtttctaaaagatggtcactggctcttcccaccgagaccagaggacagtttatccatgatttagccaccaccatgctgacctgttcacttgtccagttactttgtccagttgttggggttcatgtgtacggcaggatgtgagtcctgtacgcctgagttctgaccctatctcatcttgctaggcacatccttctgaaactactgagattccaactgtgaaatatctgtaatgagtagctggtgggaaaaatttgatgctttttaaagatgaaatttggacaaagaaagtctttaatgattagtatactgcgacatacttagtaccacatgtcgctacttattctgtagacctgcctagtgtaccttatcgcctagaaccttgaagccgctttggaagatgtatcacacgtgtttttagtcacttaagtcactagtgtttaaaaaaaaaatgtactcttcacttgggaagtatttcacgtttgttagcatgcagcttgagccttacaggcgattctggagacaagtggagcaagcttttattgcagtgtgtagtgagtcttaggcctcctttttgcctgtgcatcctaaggctagagtcattatgtgttagtgcgttagaattccttcaaacacgaaacaagctccacaacgcagtgtttctaagcatgccgtgaagaaccgagttccatgtatgacttgcccttaccagccactcttcacgcttgcagtcacgtagtacatacgtgtttacggagttcattatgtttacagattaagcgaatttctgtagttgcatttttatatttttagtatcacgttagtataaaaatttgtttaaaataaccaaagagtagttcaatgcataatttgtatgaatttgttaccaagtttcttatgctttctaaaaaaatactgtttcctatgaaaattatgtttaatcaaaagtcaagaacccttggggcaaaatgctacaggtggagtcccaccatagtcacctttgaggcacaggaagaggttccatgcatgaatctgcacacatgagcagtgctggctgctgtgcaccgggcccttgggatgaagccatcagcactgacatccagccccgtcttcatcctcaggcttagaaacaagtgggatggtgactttacagtcagtgggaagtaagagcccgcagaataacaaaggcagaaggacaggaaagcgaggtccgagtccgactgcgaggtcatatctgccaaggctccttctagtcagcttacactggtgattatacggaaaatgctcttagatatgagaaaagcaatgtggcatgtaatgagttagtgtaagcttgtgttataaactttccgaatgcttataagtagcctgtgggagctgtaagaaatacttggggccattgatttaagcttgttggctctaacatgactaatacctgtcagcccatggtagggatttcagtgacagtagtggaaacgctttccacagaccacaccactatgttgatcggtgaaagacacccccaggccagtgcatgcttccagagtgtcctgatcagtcagccaggcatggccaccgtccagtgttgatttttatcattagccagctgagcctgcagcacggggctcatttccccgtctggcagatgtggatagctgggcccactcattctgtctctcaaggctatggtaagttcttaaaggggtacttttctgcaagccaccagagtctgagtcagctgtcccctcccagtaagcatgttcacctaaagtgatttagcctaggcgaggaggatccctactacgtgctggattatgtgtgtgttcatgtaaggatttcagcgtcaaaagttcacttcctccatagtggcaaaataacactaatatgggctggtgtgttatctggagtgcccccattgtgtggaactaagggtgaaaacagaactgtacacggttgttcagtgtcgtccccagaactcatccccagcagggcttagaaaacacgagcttccttcctccttcacactcattttcctttggatgattcagtgacatcacagaacctggtgtccccctccctaccccaggtgtgtagtaatggtccgaagggaccctggctggaacaccacagccccccatgctagtgcatgaaaacatctcgttcatattctaccactggggcattgtggagtcacattcacgaaacacgaagaccttgtacttctctagaaaagtgttttagcctactttttctctaatccccaccccaacaaatgaaacacaaaattgcattacaaaagcaaacccaaatggtatttgttttcgattttatttataccgctcgagttattccttatacgtatcaagttaaactcaattggttttaatgataagctatttgacattgtttttaaattttttcttatatggtaaatgtatatccagattaaggtatcgaaatttattgcataaactataaaatcattttcaaaatctcaattccacaaataaagttctattctgattttaaaaac

SEQ ID NO: 4 - protein, 887 aa, Mus musculus
MSSDMAADESEAPVLSEDEVWEFCLDKTEDGGGSPGSDVTDTCEPPCGCWELNPNSLEEEHVLFTADPYLELHNDDTRVVRVKVIAGIGLAKKDILGASDPYVRVTLYDPMSGILTSVQTKTIKKSLNPKWNEEILFRVLPQRHRILFEVFDENRLTRDDFLGQVDVPLYPLPTENPRMERPYTFKDFVLHPRSHKSRVKGYLRLKMTYLPKNGSEDENADQAEELEPGWVVLDQPDAATHLPHPPEPSPLPPGWEERQDVLGRTYYVNHESRRTQWKRPSPDDDLTDEDNDDMQLQAQRAFTTRRQISEDVDGPDNRESPENWEIVREDENTEYSGQAVQSPPSGHIDVQTHLAEEFNTRLAVCGNPATSQPVTSSNHSSRGGSLQTCIFEEQPTLPVLLPTSSGLPPGWEEKQDDRGRSYYVDHNSKTTTWSKPTMQDDPRSKIPAHLRGKTDSNDLGPLPPGWEERTHTDGRVFFINHNIKKTQWEDPRLQNVAITGPAVPYSRDYKRKYEFFRRKLKKQTDIPNKFEMKLRRANILEDSYRRIMGVKRADLLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLQKLITLHDMESVDSEYYSSLRWILENDPTELDLRFIIDEELFGQTHQHELKTGGSEIVVTNKNKKEYIYLVIQWRFVNRIQKQMAAFKEGFFELIPQDLIKIFDENELELLMCGLGDVDVNDWREHTKYKNGYSMNHQVIHWFWKAVWMMDSEKRIRLLQFVTGTSRVPMNGFAELYGSNGPQSFTVEQWGTPDKLPRAHTCFNRLDLPPYESFDELWDKLQMAIENTQGFDGVD